(19) United States
(12) Patent Application Publication
(10) Pub. No.: US 2026/0077168 A1
(43) Pub. Date: Mar. 19, 2026

(54) METHODS, DEVICES, AND COMPONENTS FOR REGULATING FLUID FLOW

(71) Applicant: Wayne State University, Detroit, MI (US)

(72) Inventors: Carolyn Harris, Grosspointe Farms, MI (US); Ahmad Faryami, Farmington Hills, MI (US); Adam Menkara, Ontario (CA); Daniel Viar, Rochester Hills, MI (US)

(73) Assignee: Wayne State University, Detroit, MI (US)

(21) Appl. No.: 19/110,035

(22) PCT Filed: Mar. 15, 2023
(86) PCT No.: PCT/US2023/064427

§ 371 Date: Mar. 7, 2025

Related U.S. Application Data
(63) Continuation-in-part of application No. PCT/US2022/076230, filed on Sep. 9, 2022.

(51) Int. Cl. A61M 27/00; A61M 39/22

(52) U.S. Cl. CPC A61M 27/006; A61M 39/22; A61M 2027/004; A61M 2039/226; A61M 2205/0272; A61M 2205/3303; A61M 2205/3368; A61M 2205/3584; A61M 2205/50

(57) ABSTRACT

Systems and methods for regulating fluid flow are provided. Specific examples are device or system (such as a catheter system) for regulating fluid flow including a tubing configured to allow fluid flow therethrough, an implantable valve, and an external actuating component. The implantable valve includes a regulating element arranged proximate to the tubing. In use, the actuating component (which may be external to the body of the subject or arranged inside the subject's body) is arranged proximate to the regulating element. The actuating component, which may be an external actuating component, is configured to manipulate a parameter associated with the regulating element.

### CROSS-REFERENCE TO RELATED APPLICATIONS

This is the U.S. National Stage of PCT/US2023/064427, filed Mar. 15, 2023; which claims priority to and the benefit of the earlier filing of PCT Application No. PCT/US2022/076230, filed on Sep. 9, 2022, which is incorporated by reference herein in its entirety.

### STATEMENT REGARDING FEDERALLY SPONSORED RESEARCH OR DEVELOPMENT

This invention was made with government support under Grant No. 5R01NS094570-05 awarded by the National Institutes of Health (National Institute of Neurological Disorders and Stroke; NINDS). The government has certain rights in the invention.

### FIELD OF THE DISCLOSURE

This disclosure relates to regulated ventricular catheters with active outflow regulator, and other embodiments, in the context of regulating flow of biological fluids in organs or tissues, such as cerebrospinal fluid in brain tissue, and/or blood in the circulatory system. It further relates to improvements in hydrocephalous shunting systems, system elements (such as catheters, valves, actuators), and methods related to controlling fluid flow and circulation of fluids.

### BACKGROUND OF THE DISCLOSURE

Hydrocephalus, an imbalance between cerebrospinal fluid (CSF) production and absorption, is diagnosed in more than 1 in 500 people in the United States. Approximately 80% of these patients will suffer long-term neurological deficits. Genetic diseases, meningitis, subarachnoid hemorrhage, stroke, traumatic brain injury, or tumors, cause hydrocephalus.

The common treatment for all hydrocephalus patients is CSF drainage by shunting. Despite all efforts to date, shunts still have the highest failure rate of any neurological device. A shocking 98% of shunts fail after just ten years, a rate bumped up by the 80% of patients who suffer from tens if not hundreds of repetitive shunt failures. Shunts fail after becoming obstructed with attaching glia, creating a substrate for more glia or other cells and tissues (e.g. choroid plexus) to secondarily bind and block the flow of CSF through the shunt. For additional discussion, see US Patent Publication No. 2012/0060622 (Harris et al.).

Hydrocephalus patients can have a diminished quality of life and suffer from long-term neurologic deficits because of the failure of current treatments in the field, most of which involve diversion of cerebrospinal fluid (CSF) with shunts. Despite our efforts for nearly seven decades, shunts still have the highest failure rate of any neurological device: 50% of shunts fail within two years, and 98% of all shunts fail after ten years. This failure rate is the dominant contributor to the $2 billion-per-year cost that hydrocephalus incurs on our health care system.

While many factors such as infection and disconnection could lead to shunt obstruction and eventual failure, the statistics tell us that most shunts fail by becoming blocked with cells and tissues. But how does this happen? The literature predicts that there are four mechanisms: (1) cells coming from the brain, attaching and blocking the ventricular catheter; (2) cells, protein, and debris from the CSF attaching and blocking the ventricular catheter; (3) blockage by the ventricular catheter laying on the ventricular wall's epithelial cells; (4) blockage by the choroid plexus (lined with epithelial cells).

Shunts are far from ideal even when they are not occluded, and patients often experience discomfort such as headaches and pain on a regular basis which directly impact their quality of life. While valves are necessary components of a shunt system, their outdated design results in sudden pressure changes that have been shown to significantly increase the rate of shunt failure through at least three of the mechanisms mentioned earlier. In addition, regular valves offer no protection against over-drainage and under-drainage; two of the most common causes of serious conditions including but not limited to chronic headache hemorrhage.

There is an ongoing urgent need to improve hydrocephalus treatment.

### SUMMARY OF THE DISCLOSURE

This Summary is provided to introduce a selection of concepts in a simplified form that is further described below in the Detailed Description. This Summary is not intended to identify all key features or essential features of the claimed subject matter, nor is it intended to be used alone as an aid in determining the scope of the claimed subject matter.

Systems and methods for regulating fluid flow are provided in embodiments herein, which can match CSF outflow to CSF inflow (CSF production). The system provides for accounting for activity-based changes in pressure (such as sneezing, coughing, straining from constipation, exercising, and standing up), such that their impact on the outflow rate of the system is minimized or eliminated. Techniques described herein can improve patients' quality of life in the short term by stopping drainage induced headaches and in the long term by reducing the shunt failure rate.

The present disclosure provides a ventricular catheter for regulating fluid flow. In some examples, the fluid includes cerebrospinal fluid (CSF). The regulated ventricular catheter includes a tubing, a valve which is suitable to be implanted into a patient's body (which may be referred to as an “implantable valve”, or an “implanted valve” once it is implanted), and an actuating component (which in embodiments may be situated outside of the body of the patient, also referred to as “external” to their body). The tubing is configured to allow fluid flow therethrough. The implantable valve includes a regulating element arranged proximate to the tubing. The actuating component in embodiments is suitable to be arranged outside the patient's body but proximate to the regulating element. The actuating component is configured to manipulate a parameter associated with the regulating element.

In some examples, the ventricular catheter for regulating fluid flow further includes a control component, configured to communicate with other device(s) and to control the actuating component.

In some examples, the ventricular catheter further includes a housing, configured to accommodate at least a part of the tubing and the implantable valve.

In some examples, the housing further includes an insulating material, configured to reduce heat conduction between the housing and an environment.

In some examples, the ventricular catheter and/or valve further includes a sensor. The sensor includes at least one of a temperature sensor, a flowrate sensor, or a pressure sensor.

In some examples, the control component is further configured to communicate with a data processing device. In some examples, the control component receives a desired value from the processing device. In some examples, the desired value includes a desired temperature.

In some examples, the control component is further configured to communicate with the other devices via Bluetooth or other wireless manners.

In some examples, the regulating element includes wax and at least one additive. In some examples, the regulating element is configured to regulate resistance to the fluid flowing through the tubing. The resistance is independent of intracranial pressure associated with a patient.

In some examples, the actuating component, which may be an external actuating component, includes an electromagnetic field generator.

In some examples, the ventricular catheter further includes a power unit configured to provide power for the external actuating component. In some examples, the power unit includes a Lithium battery.

In some examples, the ventricular catheter further includes a heating element configured to heat the regulating element.

The present disclosure provides a system for regulating fluid flow. In some examples, the fluid includes cerebrospinal fluid (CSF). In some examples, the system includes a data processing device, a tubing, an implantable valve, an external actuating component, and a control component. The data processing device is configured to collect one or more biometric indicators associated with a patient. The tubing is configured to allow fluid flow therethrough.

The external actuating component is arranged proximate to the regulating element. The actuating component is configured to manipulate a parameter associated with the regulating element. The control component is configured to communicate with other devices and to control the external actuating component.

In some examples, the control component is further configured to communicate with a data processing device. The data processing device is further configured to calculate an estimated fluid production rate based on the one or more biometric indicators associated with the patient. The data processing device is further configured to calculate a desired value based on the estimated fluid production rate. The data processing device is further configured to send the desired value to the control component. The control component is further configured to receive the desired value from the data processing device. In some examples, the desired value includes a desired temperature.

In some examples, the one or more biometric indicators associated with the patient include a heart rate, a blood pressure, a posture indicator, an exercise indicator, and/or an endogenous brain pressure.

In some examples, the data processing device includes a wearable device.

The present disclosure provides a method for regulating fluid flow. The method includes the following operations. One or more biometric indicators associated with a patient are collected. An estimated fluid production rate based on the one or more biometric indicators associated with the patient is calculated. A desired value based on the estimated fluid production rate is calculated. The desired value is sent to a control component which controls an actuating component for actuating an implantable valve.

Examples of the method further include the following operations. The control component receives the desired value from the data processing device. The control component controls the external actuating component to actuate the regulating element. A sensor associated with the implantable valve senses a parameter associated with the regulating element. The control component determines that the parameter associated with the regulating element is within a threshold range of the desired value. The control component stops the external actuating component upon determining that the parameter associated with the regulating element is within a threshold range of the desired value.

The present disclosure also provides a computer-readable storage medium storing computer-readable instructions executable by one or more processors, that when executed by the one or more processors, causes the one or more processors to perform the acts of the method for regulating fluid flow as described above.

Systems, methods, and/or components described herein can be used in various circumstances. For example, valves, catheters, devices, and systems described herein can be used to treat hydrocephalus patients. In some examples, valves, catheters, devices, and systems described herein can be used downstream from the proximal (ventricular) tip of a ventriculoperitoneal shunt, a ventriculoatrial shunt, a lumbarperitoneal shunt, or the like. In some examples, valves, catheters, devices, and systems described herein can be used to deliver fluids, including fluids containing or acting as a drug to a subject-such as insulin, antibiotics, or other drugs, for instance to the bloodstream, the brain, the spinal column, interperitoneally, and so forth. By way of example, Wiegandt et al. (Pharmaceutics 13,657, 2021, doi: 10.3390/pharmaceutics13050657), Pane et al. (IEEE Transactions on Biomedical Engineering, 68 (7): 2088-2097, 2021), and Chonan et al. (Smart Mater. Struct. 6 (410): 410-414, 1997) illustrate exemplary circumstances in which drug delivery using the technologies described herein may be beneficial. In some examples, valves, catheters, devices, and systems described herein can be used in organs or body parts (such as brains, abdominal cavity, or the like) and/or artificial organs or artificial body parts (such as an implantable artificial bladder, an artificial urethral valve, or the like). Note that such uses are exemplary rather than limiting. Systems, methods, and/or components described herein can be used in other organs, body parts, or environments.

Also provided is a device for regulating fluid flow, including: a tubing, configured to allow fluid flow therethrough; an implantable valve including a regulating element, arranged proximate to the tubing, the regulating element including ferromagnetic material; and an external actuating component, arranged proximate to the regulating element, the external actuating component being configured to manipulate a parameter associated with the regulating element, wherein the actuating component is configured to be arranged external to a subject's body or configured to be arranged inside the subject's body, wherein the external actuating component includes an electromagnetic field generator. Examples of this device are configured to regulate fluid flowing from inside the subject's body to outside the subject's body; other examples are configured to regulate fluid flowing from outside the subject's body to inside the subject's body.

Another embodiment is a device for regulating fluid flow, including: a tubing, configured to allow fluid flow therethrough; an implantable valve including a regulating element, arranged proximate to the tubing; an actuating component, arranged proximate to the regulating element, the actuating component being configured to manipulate a parameter associated with the regulating element; and a control component, configured to communicate with other devices and to control the actuating component. In various implementations, the actuating component is configured to be arranged external to the subject's body, or it is configured to be arranged inside to the subject's body. The device may be configured to regulate fluid flowing from inside the subject's body to outside the subject's body; in other embodiments, it is configured to regulate fluid flowing from outside the subject's body to inside the subject's body.

Also provided are systems for regulating fluid flow, including: a data processing device, configured to collect one or more biometric indicators associated with a patient; and a tubing, configured to allow fluid flow therethrough; an implantable valve including a regulating element, arranged proximate to the tubing; an external actuating component, arranged proximate to the regulating element, the actuating component being configured to manipulate a parameter associated with the regulating element, wherein the actuating component is configured to be arranged external to a subject's body; and a control component, configured to communicate with other devices and to control the external actuating component. In various implementations, the actuating component of the system is configured to be arranged external to the subject's body, or it is configured to be arranged inside to the subject's body. The system may be configured to regulate fluid flowing from inside the subject's body to outside the subject's body; in other embodiments, it is configured to regulate fluid flowing from outside the subject's body to inside the subject's body.

Yet another embodiment is a method for regulating fluid flow, including: collecting one or more biometric indicators associated with a patient; calculating an estimated fluid production rate based on the one or more biometric indicators associated with the patient; calculating a desired value based on the estimated fluid production rate; and sending the desired value to a control component of an external actuating component for actuating an implantable valve of a device for regulating fluid flow, wherein the external actuating component is configured to be arranged external to the patient's body, the device for regulating fluid flow including a tubing, configured to allow fluid flow therethrough; and the implantable valve including a regulating element, arranged proximate to the tubing. In various implementations, the actuating component used in the method is configured to be arranged external to the subject's body, or it is configured to be arranged inside to the subject's body. The method may be carried out in order to regulate fluid flowing from inside the subject's body to outside the subject's body; in other embodiments, it is configured to regulate fluid flowing from outside the subject's body to inside the subject's body.

A computer-readable storage medium storing computer-readable instructions executable by one or more processors, that when executed by the one or more processors, causes the one or more processors to perform acts including: collecting one or more biometric indicators associated with a patient; calculating an estimated fluid production rate based on the one or more biometric indicators associated with the patient; calculating a desired value based on the estimated fluid production rate; and sending the desired value to a control component controlling an actuating component for actuating an implantable valve of a device, wherein the external actuating component is configured to be arranged external to the patient's body, the device for regulating fluid flow including a tubing, configured to allow fluid flow therethrough; and the implantable valve including a regulating element, arranged proximate to the tubing. In various implementations, the actuating component is configured to be arranged external to the subject's body, or it is configured to be arranged inside to the subject's body. The device that is influenced by the instructions may be configured to regulate fluid flowing from inside the subject's body to outside the subject's body; in other embodiments, it is configured to regulate fluid flowing from outside the subject's body to inside the subject's body.

### BRIEF DESCRIPTION OF DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1A illustrates an example valve for regulating fluid flow in accordance with implementations of this disclosure.

FIG. 1B illustrates an example valve for regulating fluid flow in accordance with implementations of this disclosure.

FIG. 1C illustrates an example valve for regulating fluid flow, where the tubing is arranged around the regulating element in a spiral manner in accordance with implementations of this disclosure.

FIG. 2 illustrates an example system including a valve for regulating biological fluid flow in accordance with implementations of this disclosure.

FIG. 3A and FIG. 3B illustrate an example process for regulating biological fluid flow in accordance with implementations of this disclosure.

FIG. 4 illustrates an in vitro system for testing and validating the valve and/or the system in accordance with implementations of this disclosure.

FIG. 5A and FIG. 5B illustrate graphs showing data when a patient changes position from standing up to laying down.

FIG. 6 illustrates a graph of the relationship of the flow rate versus the temperature of the regulating element.

FIG. 7 illustrates pictures of a catheter and blocked catheter holes.

FIG. 8 illustrates an example conventional catheter.

FIG. 9A, FIG. 9B, and FIG. 9C illustrate an example catheter in accordance with this disclosure.

FIG. 10 illustrates a horizontal head computed tomography (CT) image showing a catheter tip implanted in a patient's brain.

FIG. 11 illustrates an example shunt system in accordance with implementations of this disclosure.

FIG. 12A shows a simulation result of the hydrodynamics of flow through a catheter in accordance with implementations of this disclosure.

FIG. 12B illustrates a simulation result of the pressure contours inside the catheter in accordance with implementations of this disclosure.

FIG. 12C illustrates a simulation result of the velocity contours inside the catheter in accordance with implementations of this disclosure.

FIG. 13A and FIG. 13B illustrate an implantable valve for regulating fluid flow in accordance with implementations of this disclosure.

FIG. 14 illustrates an example system including an implantable valve and an external actuating component in accordance with implementations of this disclosure.

FIG. 15 illustrates an example earpiece in accordance with implementations of this disclosure.

FIG. 16 illustrates a diagram showing curves of flow rates versus temperature where the regulating element has different additive proportions.

FIG. 17 illustrates a curve of the time to heat the regulating element to 100° C. versus a distance between the regulating element and the actuating component.

FIG. 18 illustrates a diagram showing curves of temperatures versus time.

FIG. 19 illustrates the impact of the inner diameter of the tubing on the flow rate of the fluid flowing through the tubing.

FIG. 20A and FIG. 20B illustrate an example process for regulating biological fluid flow in accordance with implementations of this disclosure.

### DETAILED DESCRIPTION

Hydrocephalus, an imbalance between cerebrospinal fluid production and absorption, is diagnosed in more than 1 in 500 people in the United States. Approximately 80% of these patients will suffer long-term neurological deficits. Genetic diseases, meningitis, subarachnoid hemorrhage, stroke, traumatic brain injury, or tumors cause hydrocephalus. The common treatment for all hydrocephalus patients is CSF drainage by shunting.

Despite all prior efforts, shunts still have the highest failure rate of any neurological device. A shocking 98% of shunts fail after just ten years, a rate bumped up by the 80% of patients who suffer from tens if not hundreds of repetitive shunt failures. Shunts fail after becoming obstructed with attaching glia, creating a substrate for more glia or other cells and tissues (e.g. choroid plexus) to secondarily bind and block the flow of CSF through the shunt.

A regulated ventricular catheter provided herein is capable of matching CSF outflow to CSF inflow (CSF production). Activity-based changes in pressure (such as sneezing, coughing, straining from constipation, exercising, and standing up) will not impact the outflow rate of the valve. This regulated ventricular catheter can improve patients' quality of life in the short term by stopping drainage induced headaches and in the long term by reducing the shunt failure rate.

Also provided herein is a system including an implantable valve (which may be implanted in a patient's body, such as the brain ventricle) and an actuating component arranged outside (that is, external to) the patient's body. As described herein, an implant/internal/implanted component can refer to a component that is implanted into a patient's body. An external component refers to a component that is arranged outside the patient's body. The external actuating component is configured to actuate a regulating element in the internal valve to regulate the fluid flow through the implant valve.

A benchtop model testing device was used to test the impact of regular shunt valves in comparison to the described valve on shunt failure. Furthermore, the UTD can simulate changes in pressure within the head based on activities (such as standing up, jogging, or sneezing) that are typically associated with chronic headaches and accurately measure the performance of each valve against over-drainage and under-drainage.

Also provided herein is a system including the solid state valve with active outflow regulator, for instance for regulating flow of cerebral spinal fluid (CFS) or another fluid. Described improvements in various embodiments include using a regulating element (such as a formula including wax and additives) to increase/decrease resistance around catheter tubing to the valve. The regulating element may be manipulated by an actuator arranged proximate to the regulating element. The regulating element in embodiments is controlled based on an estimated CSF production rate, which can be calculated based on biometric indicators associated with the patient. Methods are described for estimating CSF production rate using an algorithm based at least on biometric indicators such as a heart rate and a blood pressure associated with the patient.

In representative embodiments, the shunt catheter design is improved in terms of the shape of the catheter and/or the hole sizes, which can be tailored to manipulate the flow velocity and shear rate/shear stress through the catheter holes and the internal lumen of the ventricular catheter.

Aspects of the current disclosure are now described in additional detail, as follows: (I) Structure and Function of Valve Embodiments; (II) Overview of Embodiments of System with Valve; (III) Methods and Operations of Controlling Valve System Embodiments; (IV) Materials of Regulating Element Embodiments; (V) Algorithm for Controlling Valve Embodiments; (VI) Kits; (VII) Catheter Design; (VIII) Structure and Function of Implantable Valve Embodiments; (IX) Overview of System Embodiments with Implantable Valve and External Actuating Component; (X) “Earpiece” Designs; (XI) Characteristics of System Embodiments; (XII) Methods and Operations of Controlling a System with Implantable Valve and Actuating Component; (XIII) Example 1; (XIV) References; (XV) Example Clauses; and (XVI) Closing Paragraphs.

### (I) Structure and Function of Valve Embodiments

As described above, CSF drainage by shunting is a common treatment for hydrocephalus patients, and can be implemented with a shunt. The shunt usually includes a ventricular catheter, a valve, and a distal catheter. In some cases, hydrocephalus patients can experience high failure rate of shunts which can lead to diminished quality of life and suffering from long-term neurologic deficits. In some instances, conventional shunt valve controls CSF outflow by total pressure within the ventricles. The conventional shunt's one-way pressure valve is set to relieve pressure from the ventricles based off of total pressure, where total pressure=hydrostatic pressure+(intracranial pressure (ICP)−intraabdominal pressure (IAP)). If the total pressure within the ventricles exceeds that of the conventional valve, the conventional valve will open. It will remain open unless the pressure drops below the set valve pressure. However, many events such as sneezing, coughing, straining from constipation, exercising, and standing up of the patient can cause changes in pressure, without increasing the production of the CSF. As a result, the conventional shunt valve can over-drain the CSF because it is pressure-based rather than matching the CSF production. Moreover, tissue contact could happen between the catheter tip and surrounding tissue because of the over-drainage. As such, the tissue can be pulled into the catheter holes and block the catheter.

Therefore, this disclosure provides a shunt valve with active outflow regulator. The valve according to this disclosure is capable of controlling the CSF outflow by regulating the resistance of the tubing which is independent of the intracranial pressure. Tissue contact can be attenuated with the valve that minimizes over-drainage by matching the CSF outflow to the physiologic CSF production. This valve offers protection against activity-based changes in pressure, and thus can improve patients' quality of life in the short term by preventing over-drainage induced headache and in the long term by reducing the shunt failure rate.

Moreover, other applications outside of hydrocephalus are also possible. For example, the valve provided by this disclosure can be used at downstream from proximal (ventricular) tip of a ventriculoperitoneal shunt, a ventriculoatrial shunt, a lumbar peritoneal shunt, or the like.

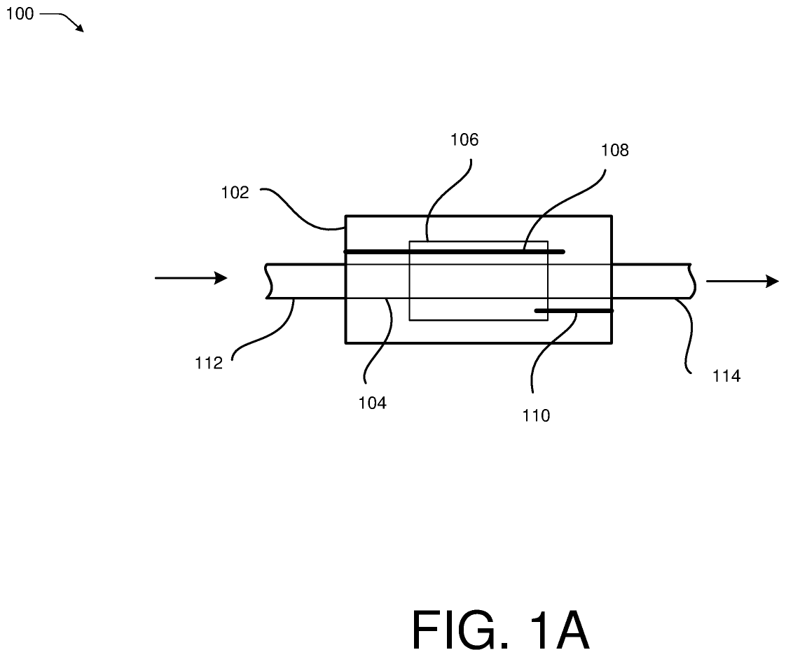

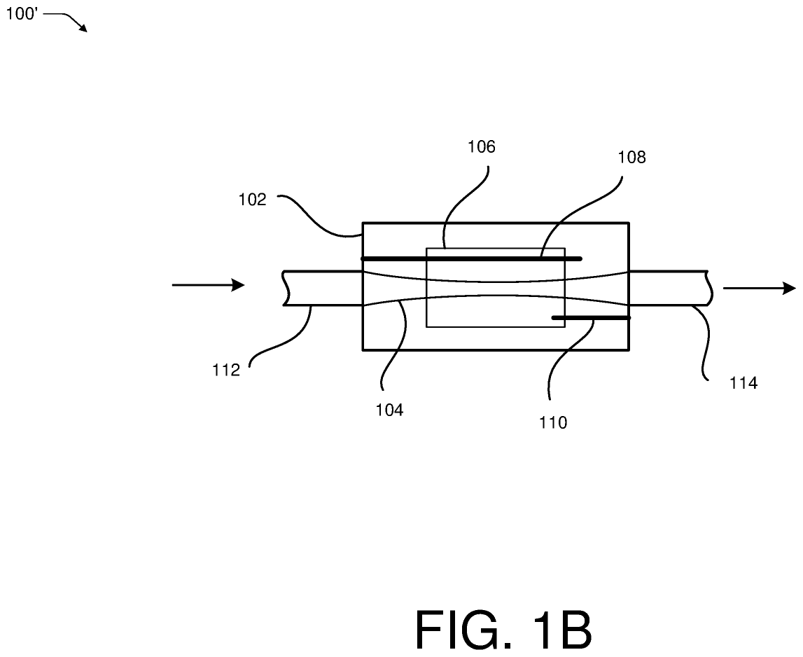

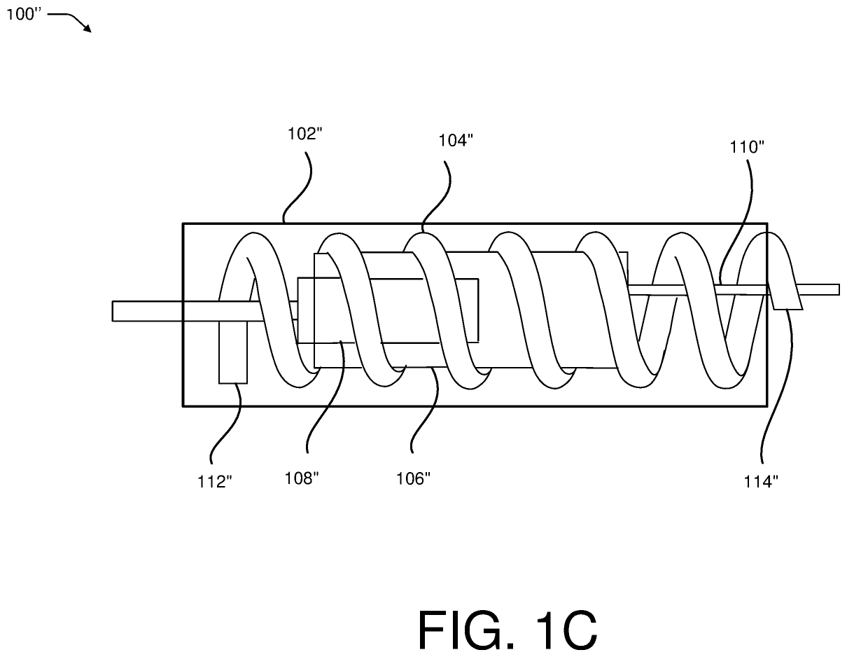

FIG. 1A illustrates an example valve 100 for regulating fluid flow in accordance with implementations of this disclosure. In some examples, the valve 100 is a solid state valve. The solid state valve can refer to a valve using materials that are not fluid but retain the solid state. In some examples, the fluid flowing through the valve 100 includes biological fluids in organs or tissues, such as cerebrospinal fluid (CSF) in brain tissue, blood in the circulatory system, and so on. In some examples, the valve 100 can be a part of a shunt system for treating hydrocephalous.

Referring to FIG. 1A, the valve 100 includes a housing 102, a tubing 104, a regulating element 106, an actuator 108, a sensor 110, and a control component (not shown). As described herein, the structure of the valve 100 is exemplary, and the valve 100 can further include other components.

The housing 102 is configured to accommodate the tubing 104, the regulating element 106, the actuator 108, and the control component. In some examples, the housing further includes an insulating element (not shown), configured to reduce heat conduction between the housing and an environment such as the ventricle. In some examples, since the valve can be implanted into the patient's brain, and the valve 100 may generate heat in use, the insulating element can inhibit the heat conduction from the valve 100 to the surrounding environment (such as brain ventricles). In some examples, the internal temperature needed for valve closure is 70° C. In some examples, the operating temperature for the valve can be 50-70° C. In some examples, the operating temperature for the valve can be 90-110° C. For examples, polyethylene which has a relatively low thermally conductivity of 0.33-0.5 W/m K can be used as the insulating element. As another example, polypropylene which has a relatively low thermally conductivity of 0.11 W/m K can be used as the insulating element.

In some examples, the housing 102 can further include a heat sink (not shown), configured to dissipate away the heat generated the valve 100, thereby decreasing of the temperature of the valve 100. Examples of heat sinks can include passive heat sinks, active heat sinks, metal heat sinks (such as aluminum heat sinks, copper heat sinks, or the like), heat sinks with coolant, or the like. Passive heat sinks are those that do not rely on forced air flow (fans). Active heat sinks have a powered device such as a fan or blower in close proximity to the heat exchanger surface.

The tubing 104 is configured to allow fluid flow therethrough. In some examples, biological fluid can flow through the tubing 104 such as cerebrospinal fluid (CSF) in brain tissue, blood in the circulatory system, and so on. In some examples, a first end 112 and a second end 114 of the tubing can connect to catheters such as a ventricular catheter, a distal catheter, or the like.

The regulating element 106 is arranged proximate to the tubing 104. In some examples, the regulating element 106 includes materials that are suitable to squeeze and/or release the tubing to regulate resistance to the fluid flowing through the tubing 104. The resistance is independent of intracranial pressure associated with a patient. For example, the regulating element 106 includes wax and at least one additive. Examples of the additive include graphite, ferromagnetic material (e.g., iron oxide, or the like), and the like. The graphite can be added to the wax to adjust the melting temperature of the regulating element 106. When there is an applied electromagnetic field, the ferromagnetic material in the regulating element can generate an induced electromagnetic field to generate heat to raise the temperature of the regulating element 106. In some examples, the ferromagnetic material can be used to form powders, beads, irregular pieces, or the like to be added to the wax. In some examples, the ferromagnetic material can take the form of electroplating. In implementations, the mass of the ferromagnetic material in the regulating element 106 can be limited such that the ferromagnetic material would produce little impact on MRI (e.g., small artifacts without long-term manipulation of the MRI results). As an example, for wax with a volume of 2 mL, the mass of the ferromagnetic material add can be 0.1 to 0.6 gram (g). Additional details regarding the materials of the regulating element are described throughout this disclosure, such as in section (V).

The actuator 108 is configured to manipulate a parameter associated with the regulating element. In some examples, the parameter associated with the regulating element can be the temperature associated with the regulating element. The actuator 108 is arranged proximate to the regulating element. In some examples, the actuator 108 includes a temperature actuator such as a heating element. Note that other types of actuators can also be used in the valve 100 such as mechanical actuators, electro-mechanical actuators, or the like. In some examples, the actuator 108 can include an electromagnetic generator (e.g., a coil, a magnetic solenoid, or the like), configured to apply an electromagnetic field to the valve 102. In implementations, since the valve can be implanted into a brain of a patient, it is preferable that the actuator 108 is magnetic resonance imaging (MRI) compatible such that when the patient goes through an MRI procedure, the actuator 108 would not interfere with the procedure.

The sensor 110 is configured to sense the parameter associated with regulating element 106. In some examples, the sensor 110 includes a temperature sensor. In some instances, the sensor 110 is arranged proximate to the regulating element 106. Note that other types of sensors can be used to detect parameters associated with the fluid flowing through the valve 100, such as a flowrate sensor, or a pressure sensor, or the like. In some instances, sensor 110 is arranged proximate to the tubing 104 or inside the tubing 104.

The control component (not shown) is configured to communicate with other devices and to control the actuator 108. In some examples, the control component can communicate with the other devices via networks such as the Internet, a Mobile Telephone Network (MTN), Wi-Fi, a cellular network (e.g., 2G, 3G, 4G, 4G LTE, 5G, etc.), a mesh network, a Local Area Network (LAN), a Wide Area Network (WAN), a Virtual LAN (VLAN), a private network, short range wireless frequencies such as Bluetooth, and/or other various wired or wireless communication technologies. For example, the control component can communicate with a data processing device (such as a wearable device) to receive data or instructions useful for controlling the valve 100.

The control component can control the actuator 108 to actuate the regulating element. For example, the control component can receive a desired value (such as a desired temperature) from other devices (such as the data processing device). The sensor 110 can sense the parameter (such as the temperature) associated with the regulating element 106. The control component can determine that the parameter (such as the temperature) associated with the regulating element 106 is within a threshold range of the desired value (such as the desired temperature). The control component can stop the actuator 108 from actuating upon determining that the parameter (such as the temperature) associated with the regulating element 106 is within a threshold range of the desired value (such as the desired temperature). The threshold range can be set arbitrarily.

The valve 100 further includes a power unit (not shown), configured to provide power for the valve 100. In some instances, the power unit includes a Lithium battery. In some examples, the power unit can be percutaneous recharged.

FIG. 1B illustrates an example valve 100′ for regulating fluid flow in accordance with implementations of this disclosure. The valve 100′ has the same structure and function as the valve 100. The same reference number refer to the same element/component.

Referring to FIG. 1B, the tubing 104 is quizzed due to the deformation of the regulating element 106. Similar to the valve 100, the valve 100′ receives a desired value (such as a desired temperature) from a data processing device. Then, the control component (not shown) controls the actuator 108 to heat the regulating element 106 to the desired value. As an example, the regulating element 106 includes materials (such as wax and additives) that melts when the temperature increases and compresses the tubing 104. As such, the fluid flow (such as CSF flow) through the tubing 104 is reduced. The sensor 110 can sense the temperature associated with the regulating element 106. When the control component determines that the temperature associated with the regulating element 106 is within a threshold range of the desired value (such as the desired temperature), the control component can stop the actuator 108 from heating the regulating element 106.

FIG. 1C illustrates an example valve 100″ for regulating fluid flow, where the tubing 104″ is arranged around the regulating element 106″ in a spiral manner in accordance with implementations of this disclosure. In some examples, the valve 100″ is a solid state valve. In some examples, the fluid flowing through the valve 100″ includes biological fluids in organs or tissues, such as cerebrospinal fluid (CSF) in brain tissue, blood in the circulatory system, and so on. In some examples, the valve 100″ can be a part of a shunt system for treating hydrocephalous.

Referring to FIG. 1A, the valve 100″ includes a housing 102″, a tubing 104″, a regulating element 106″, an actuator 108″, a sensor 110″, and a control component (not shown). As described herein, the structure of the valve 100″ is exemplary, and the valve 100″ can further include other components.

The housing 102″ is configured to accommodate the tubing 104″, the regulating element 106″, the actuator 108″, and the control component. In some examples, the housing further includes an insulating element (not shown), configured to reduce heat conduction between the housing and an environment such as the ventricle. In some examples, since the valve can be implanted into the patient's brain, and the valve 100″ may generate heat in use, the insulating element can inhibit the heat conduction from the valve 100″ to the surrounding environment (such as brain ventricles). In some examples, the internal temperature needed for valve closure is 70° C. For examples, polyethylene which has a relatively low thermally conductivity of 0.33-0.5 W/m K can be used as the insulating element. As another example, polypropylene which has a relatively low thermally conductivity of 0.11 W/m K can be used as the insulating element.

The tubing 104″ is configured to allow fluid flow therethrough. In some examples, biological fluid can flow through the tubing 104″ such as cerebrospinal fluid (CSF) in brain tissue, blood in the circulatory system, and so on. In some examples, a first end 112″ and a second end 114″ of the tubing can connect to catheters such as a ventricular catheter, a distal catheter, or the like.

The regulating element 106″ is arranged proximate to the tubing 104″. In some examples, the regulating element 106″ includes materials that are suitable to squeeze and/or release the tubing to regulate resistance to the fluid flowing through the tubing 104″. The resistance is independent of intracranial pressure associated with a patient. For example, the regulating element 106″ includes wax and at least one additive. Examples of the additive include graphite, ferromagnetic material (e.g., iron oxide, or the like), and the like. The graphite can be added to the wax to adjust the melting temperature of the regulating element 106″. When there is an applied electromagnetic field, the ferromagnetic material in the regulating element can generate an induced electromagnetic field to generate heat to raise the temperature of the regulating element 106″. In some examples, the ferromagnetic material can be used to form powders, beads, irregular pieces, or the like to be added to the wax. In some examples, the ferromagnetic material can take the form of electroplating. In implementations, the mass of the ferromagnetic material in the regulating element 106″ can be limited such that the ferromagnetic material would produce little impact on MRI (e.g., small artifacts without long-term manipulation of the MRI results). As an example, for wax with a volume of 2 mL, the mass of the ferromagnetic material add can be 0.1 to 0.6 gram (g). Additional details regarding the materials of the regulating element are described throughout this disclosure, such as in section (V).

The actuator 108″ is configured to manipulate a parameter associated with the regulating element. In some examples, the parameter associated with the regulating element can be the temperature associated with the regulating element. The actuator 108″ is arranged proximate to the regulating element. In some examples, the actuator 108″ includes a temperature actuator such as a heating element. Note that other types of actuators can also be used in the valve 100″ such as mechanical actuators, electro-mechanical actuators, or the like. In some examples, the actuator 108″ can include an electromagnetic generator (e.g., a coil, a magnetic solenoid, or the like), configured to apply an electromagnetic field to the valve 202. In implementations, since the valve can be implanted into a brain of a patient, it is preferable that the actuator 108″ is magnetic resonance imaging (MRI) compatible such that when the patient goes through an MRI procedure, the actuator 108″ would not interfere with the procedure.

The sensor 110″ is configured to sense the parameter associated with regulating element 106″. In some examples, the sensor 110″ includes a temperature sensor. In some instances, the sensor 110″ is arranged proximate to the regulating element 106″. Note that other types of sensors can be used to detect parameters associated with the fluid flowing through the valve 100″, such as a flowrate sensor, or a pressure sensor, or the like. In some instances, sensor 110″ is arranged proximate to the tubing 104″ or inside the tubing 104″.

The control component (not shown) is configured to communicate with other devices and to control the actuator 108″. In some examples, the control component can communicate with the other devices via networks such as the Internet, a Mobile Telephone Network (MTN), Wi-Fi, a cellular network (e.g., 2G, 3G, 4G, 4G LTE, 5G, etc.), a mesh network, a Local Area Network (LAN), a Wide Area Network (WAN), a Virtual LAN (VLAN), a private network, short range wireless frequencies such as Bluetooth, and/or other various wired or wireless communication technologies. For example, the control component can communicate with a data processing device (such as a wearable device) to receive data or instructions useful for controlling the valve 100″.

The control component can control the actuator 108″ to actuate the regulating element. For example, the control component can receive a desired value (such as a desired temperature) from other devices (such as the data processing device). The sensor 110″ can sense the parameter (such as the temperature) associated with the regulating element 106″. The control component can determine that the parameter (such as the temperature) associated with the regulating element 106″ is within a threshold range of the desired value (such as the desired temperature). The control component can stop the actuator 108″ from actuating upon determining that the parameter (such as the temperature) associated with the regulating element 106″ is within a threshold range of the desired value (such as the desired temperature). The threshold range can be set arbitrarily.

The valve 100″ further includes a power unit (not shown), configured to provide power for the valve 100″. In some instances, the power unit includes a Lithium battery. In some examples, the power unit can be percutaneous recharged.

As an example, the tubing 104″ can be quizzed due to the deformation of the regulating element 106″. Similar to the valve 100, the valve 100″ receives a desired value (such as a desired temperature) from a data processing device. Then, the control component (not shown) controls the actuator 108″ (such as a heating element) to heat the regulating element 106″ to the desired value. As an example, the regulating element 106″ includes materials (such as wax and additives) that melts when the temperature increases and compresses the tubing 104″. As such, the fluid flow (such as CSF flow) through the tubing 104″ is reduced. The sensor 110″ can sense the temperature associated with the regulating element 106″. When the control component determines that the temperature associated with the regulating element 106″ is within a threshold range of the desired value (such as the desired temperature), the control component can stop the actuator 108″ from heating the regulating element 106.

### (II) Overview of Embodiments of System with Valve

This disclosure also provides a system including the valve for regulating biological fluid flow as described above. In some instances, the system can be used to treat hydrocephalous patients. At least a part of the system can be implanted into a patient's brain. Such a system offers protection against activity-based changes in pressure, and is specifically engineered to improve patients' quality of life in the short term by stopping drainage induced headache and in the long term by reducing the shunt failure rate. In some instances, the system can also be used to conduct in vitro experiments, to collect analytic data, to validate shunt valve functions, etc.

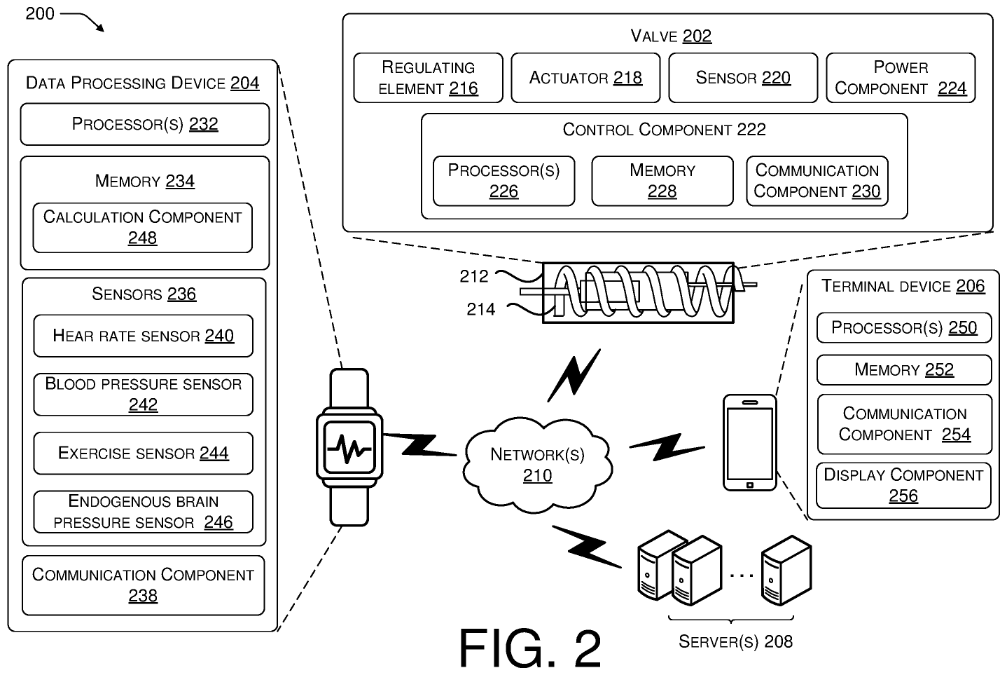

FIG. 2 illustrates an example system 200 including a valve 202 for regulating biological fluid flow in accordance with implementations of this disclosure. Referring to FIG. 2, the system 200 includes a valve 202 for regulating fluid flow, a data processing device 204, a terminal device 206, and a server 208. In implementations, the data processing device 204, the valve 202 for regulating fluid flow, the terminal device 206, and the server 208 can communicate with each other wired or wirelessly via one or more networks 210. For example, the network(s) 210 can include the Internet, a Mobile Telephone Network (MTN), Wi-Fi, a cellular network (e.g., 2G, 3G, 4G, 4G LTE, 5G, etc.), a mesh network, a Local Area Network (LAN), a Wide Area Network (WAN), a Virtual LAN (VLAN), a private network, short range wireless frequencies such as Bluetooth, and/or other various wired or wireless communication technologies.

The valve 202 for regulating fluid flow can be the same as the valve 100, the valve 100′, and/or the valve 100″ as described with respect to FIG. 1A, FIG. 1B, and FIG. 1C. In some examples, the valve 202 is a solid state valve. In some examples, the fluid flowing through the valve 100 includes biological fluids in organs or tissues, such as cerebrospinal fluid (CSF) in brain tissue, blood in the circulatory system, and so on.

The valve 202 includes a housing 212, a tubing 214, a regulating element 216, an actuator 218, a sensor 220, and a control component 222. As described herein, the structure of the valve 202 is exemplary, and the valve 202 can further include other components.

The housing 212 is configured to accommodate the tubing 214, the regulating element 216, the actuator 218, and the control component 222. In some examples, the housing further includes an insulating element (not shown), configured to reduce heat conduction between the housing and an environment such as the ventricle. In some examples, since the valve can be implanted into the patient's brain, and the valve 202 may generate heat in use, the insulating element can inhibit the heat conduction from the valve 202 to the surrounding environment (such as brain ventricles). In some examples, the internal temperature needed for valve closure is 70° C. For examples, polyethylene which has a relatively low thermally conductivity of 0.33-0.5 W/m K can be used as the insulating element. As another example, polypropylene which has a relatively low thermally conductivity of 0.11 W/m K can be used as the insulating element. Note that any material that is suitable for inhibiting the heat conduction from the valve 202 to the surrounding environment (such as brain ventricles) can be used as the insulating element.

The tubing 214 is configured to allow fluid flow therethrough. In some examples, biological fluid can flow through the tubing 214 such as cerebrospinal fluid (CSF) in brain tissue, blood in the circulatory system, and so on. In some examples, the tubing 214 can connect to catheters such as a ventricular catheter, a distal catheter, or the like.

The regulating element 216 is arranged proximate to the tubing 214. In some examples, the regulating element 216 includes materials that are suitable to squeeze and/or release the tubing to regulate resistance to the fluid flowing through the tubing 214. The resistance is independent of intracranial pressure associated with a patient. For example, the regulating element 216 includes wax and at least one additive (such as graphite and the like). Additional details regarding the materials of the regulating element are described throughout this disclosure, such as in section (V).

The actuator 218 is configured to manipulate a parameter associated with the regulating element 216. In some examples, the parameter associated with the regulating element 216 can be the temperature associated with the regulating element 216. The actuator 218 is arranged proximate to the regulating element 216. In some examples, the actuator 218 includes a temperature actuator such as a heating element. Note that other types of actuators can also be used in the valve 202 such as mechanical actuators, electro-mechanical actuators, or the like. In implementations, since the valve 202 can be implanted into a brain of a patient, it is preferable that the actuator 118 is magnetic resonance imaging (MRI) compatible such that when the patient goes through an MRI procedure, the actuator 118 would not interfere with the procedure.

The sensor 220 is configured to sense the parameter associated with regulating element 216. In some examples, the sensor 220 includes a temperature sensor. In some instances, the sensor 220 is arranged proximate to the regulating element 216. Note that other types of sensors can be used to detect parameters associated with the fluid flowing through the valve 202, such as a flowrate sensor, or a pressure sensor, or the like. In some instances, sensor 220 is arranged proximate to the tubing 214 or inside the tubing 214.

The control component 222 is configured to communicate with other devices and to control the actuator 218. In some examples, the control component 222 can communicate with the other devices via network(s) 210. For example, the control component 222 can communicate with the data processing device (such as a wearable device) 204 to receive data or instructions useful for controlling the valve 202.

The control component 222 can control the actuator 218 to actuate the regulating element 216. For example, the control component 222 can receive a desired value (such as a desired temperature) from the data processing device 204. The sensor 220 can sense the parameter (such as the temperature) associated with the regulating element 216. The control component 222 can determine that the parameter (such as the temperature) associated with the regulating element 216 is within a threshold range of the desired value (such as the desired temperature). The control component 222 can stop the actuator 218 from actuating upon determining that the parameter (such as the temperature) associated with the regulating element 216 is within a threshold range of the desired value (such as the desired temperature). The threshold range can be set arbitrarily. As an example, the regulating element 216 includes materials (such as wax and additives) that melts when the temperature increases and compresses the tubing 214. As such, the fluid flow (such as CSF flow) through the tubing 214 is reduced. The sensor 220 can keep sensing the temperature associated with the regulating element 216. When the control component 222 determines that the temperature associated with the regulating element 216 is within a threshold range of the desired value (such as the desired temperature), the control component 222 can stop the actuator 218 from heating the regulating element 106.

The valve 202 further includes a power unit 224, configured to provide power for the valve 202. In some instances, the power unit 224 includes a Lithium battery. In some examples, the power unit 224 can be percutaneous recharged.

The control component 222 includes one or more processors 226, a memory 228, a communication component 230. The processor(s) 226 can be a single processing unit or a number of units, each of which could include multiple different processing units. The processor(s) 00 can include a microprocessor, a microcomputer, a microcontroller, a digital signal processor, a central processing unit (CPU), a graphics processing unit (GPU), a security processor etc. Alternatively, or in addition, some or all of the techniques described herein can be performed, at least in part, by one or more hardware logic components. For example, and without limitation, illustrative types of hardware logic components that can be used include a Field-Programmable Gate Array (FPGA), an Application-Specific Integrated Circuit (ASIC), an Application-Specific Standard Products (ASSP), a state machine, a Complex Programmable Logic Device (CPLD), pulse counters, resistor/coil readers, other logic circuitry, a system on chip (SoC), and/or any other devices that perform operations based on instructions. Among other capabilities, the processor(s) 226 can be configured to fetch and execute computer-readable instructions stored in the memory.

The memory 228 can include one or a combination of computer-readable media. As used herein, “computer-readable media” includes computer storage media and communication media. Computer storage media includes volatile and non-volatile, removable and non-removable media implemented in any method or technology for storage of information, such as computer-readable instructions, data structures, program modules, or other data. Computer storage media includes, but is not limited to, Phase Change Memory (PRAM), Static Random-Access Memory (SRAM), Dynamic Random-Access Memory (DRAM), other types of Random-Access Memory (RAM), Read Only Memory (ROM), Electrically Erasable Programmable ROM (EEPROM), flash memory or other memory technology, Compact Disk ROM (CD-ROM), Digital Versatile Disks (DVD) or other optical storage, magnetic cassettes, magnetic tape, magnetic disk storage or other magnetic storage devices, or any other medium that can be used to store information for access by a computing device. In contrast, communication media includes computer-readable instructions, data structures, program modules, or other data in a modulated data signal, such as a carrier wave. As defined herein, computer storage media does not include communication media.

The memory 228 can include an operating system configured to manage hardware and services for the benefit of other components and devices.

The communication component 230 is configured to communicate with other devices (e.g., in the data processing device 204, the terminal device 206, the server(s) 208, and so on) the network(s) 210. For example, the communication component 230 can perform compression, encryption, and/or formatting of the data received and/or generated by the sensors 236. In some embodiments, the communication component 230 can transmit data using one or more protocols or languages, such as an extensible markup language (XML), Modbus, HTTP, HTTPS, USB, etc.

The data processing device 204 is configured to collect one or more biometric indicators associated with a patient. In some examples, the one or more biometric indicators associated with the patient include a heart rate, a blood pressure, a posture indicator, an exercise indicator, and/or an endogenous brain pressure. In some examples, data processing device 204 includes, but is not limited to, smart watches, smart accessories (e.g., smart rings, smart wristbands, smart pins, smart bracelets, or the like), body-mounted sensors, fitness trackers, smart clothing (e.g., smart shirt, smart pants, smart belt, or the like), smart headband, smart headsets, smart footwears, smart glasses, or the like.

The data processing device 204 includes one or more processors 232, a memory 234, one or more sensors 236, and a communication component 238. The processor(s) 232, the memory 234, and the communication component 238 can be implemented in the same manner as the processor(s) 226, the memory 228, and the communication component 230.

The sensor(s) 236 include a heart rate sensor 240, a blook pressure sensor 242, an exercise sensor 244, endogenous brain pressure sensor 246 (such as a telemetric Intracranial pressure sensors), and other sensors suitable for detecting biometric indicators associated with the patient.

The data processing device 204 is further configured to calculate an estimated fluid production rate based on the one or more biometric indicators associated with the patient. The memory 234 includes a calculation component 248 storing computer-readable instructions executable by one or more processors, that when executed by the one or more processors, causes the one or more processors to perform acts. For example, the calculation component 248 can implement algorithms to calculate an estimated fluid production rate based on the one or more biometric indicators associated with the patient. The calculation component 248 can implement algorithms to calculate a desired value based on the estimated fluid production rate. Additional details of algorithms are provided throughout this disclosure such as in section (VI).

The data processing device 204 is further configured to send the desired value to the control component 222 of the valve 202.

The terminal device 206 includes one or more processor(s) 250, a memory 252, and a communication component 254, each of which can be implemented similar to the processor(s) 226, the memory 228, and/or the communication component 230 of the control component 222 of the valve 202. Furthermore, the terminal device 206 can include a display component 256 configured to display a graphical user interface on the terminal device 206.

The terminal device 206 can run one or more applications (Apps) to facilitate user interaction via the user interface and/or collect data. In some examples, the terminal device 206 can generate reports and present the reports via the user interface. For example, the reports can include information like the patient identification (ID), shunt ID, biometric indicators associated with the patient, settings associated with the valve, or the like. In some examples, the terminal device 206 can provide interactive functionality (e.g., input boxes, dropdown lists, selectable fields, or the like) via the user interface to allow the user to change settings of the valve.

The terminal device 206 can perform security functions. For example, terminal device 206 can be configured to perform multi-factor authentication (such as two-factor authentication, or the like) when a user is logging in to the App. Multi-factor authentication is an authentication method in which a user is granted access to a website or application after successfully presenting two or more pieces of evidence to an authentication mechanism such as passwords, security question answers, verification codes, or the like.

A user (e.g., a patient, a care giver, a physician, or the like) can interact with the terminal device 206 via the user interface to perform a variety of operations. In some examples, the user can review reports generated by the terminal device 206, the biometric indicators provided by the data processing device 204, or the like. In some examples, the user can make changes to the settings of the valve 202 via the user interface.

The server(s) 208 is configured to perform back-end control over other devices, such as providing security functions, updating software and/or algorithms, or the like. In some examples, the server(s) 208 can include computing devices that operate within a network service (e.g., a cloud service), or can form a mesh network, etc. In some examples, the servers 208 is configured to communicate with the terminal device to perform security function. For example, the servers 208 can store information like user ID, shunt ID, authentication information (e.g., passcodes, security questions and answers, fingerprint features, facial features, or the like). When the user is logging in an App on the terminal device 206, the terminal device 206 can comminate with the server(s) 208 to get the authentication information to verify the user's identity.

The servers 208 is configured to update software and/or algorithms on different devices such as the control component, the data processing device 00, the terminal device 00, or the like. In implementations, the software and/or algorithms can be improved to provide more accurate calculation of the CSF production, more precise control over the value, or the like. Therefore, it may be beneficial to update the software and/or algorithms constantly.

The techniques discussed above can be implemented in hardware, software, or a combination thereof. In the context of software, operations represent computer-executable instructions stored on one or more computer-readable storage media that, when executed by one or more processors, configure a device to perform the recited operations. Generally, computer-executable instructions include routines, programs, objects, components, data structures, and the like that perform particular functions or implement particular abstract data types.

### (III) Methods and Operations of Controlling Valve System Embodiments

Methods and operations described herein can be used to control the system as described above to regulate biological fluid flow (e.g., CSF, or the like). In some examples, methods and operations described herein can be implemented to match the CSF outflow of a valve system to CSF inflow (CSF production) associated with a patient. Activity-based changes in pressure such as a sneezing, coughing, and standing up will not impact the outflow rate of the valve. In some examples, methods and operations described herein can be used to treat hydrocephalous patients, improving patients' quality of life in the short term by stopping drainage induced headache and in the long term by reducing the shunt failure rate.

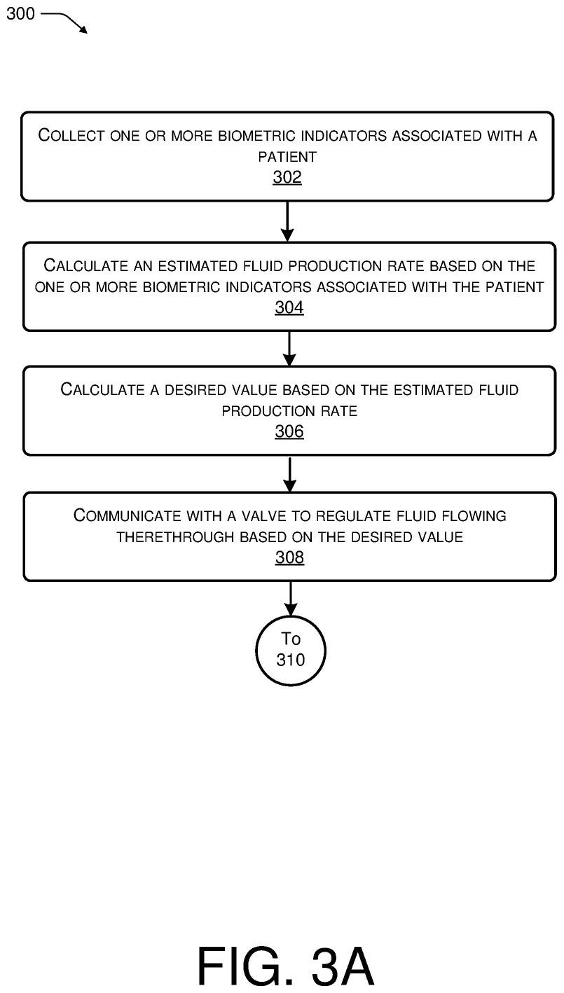

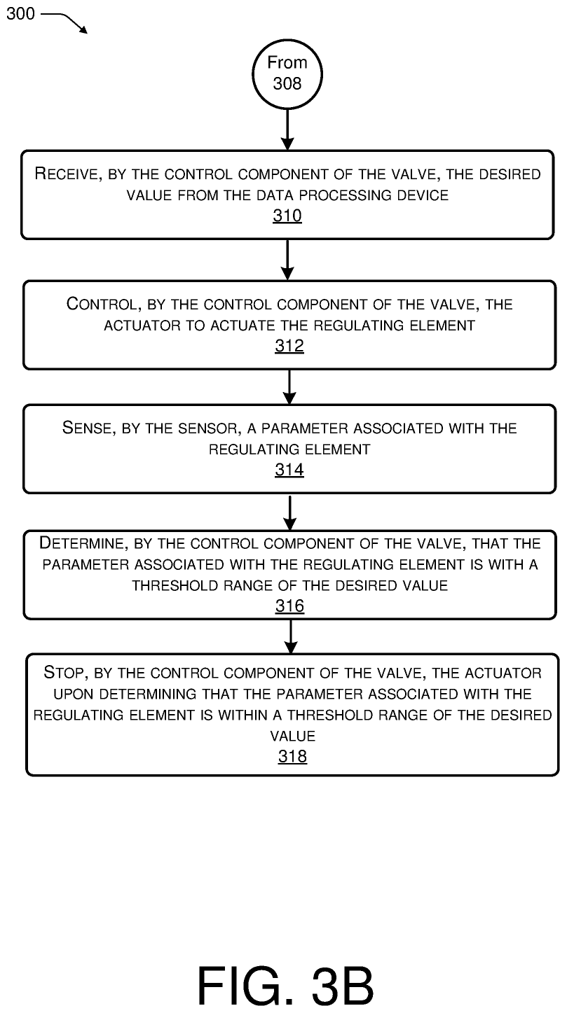

The example processes (e.g., in FIGS. 3A and 3B) are illustrated as logical flow graphs, each operation of which represents a sequence of operations that can be implemented in hardware, software, or a combination thereof. In the context of software, the operations represent computer-executable instructions stored on one or more computer-readable storage media that, when executed by one or more processors, configure a device to perform the recited operations. Generally, computer-executable instructions include routines, programs, objects, components, data structures, and the like that perform particular functions. The order in which the operations are described is not intended to be construed as a limitation, and any number of the described operations can be combined in any order and/or in parallel to implement the process. Further, any of the individual operations can be omitted, repeated, reordered, and/or combined with other operations.

FIG. 3A and FIG. 3B illustrate an example process 300 for regulating biological fluid flow in accordance with implementations of this disclosure.

Referring to FIG. 3A, at 302, operations include collecting one or more biometric indicators associated with a patient. In some examples, the one or more biometric indicators associated with the patient include a heart rate, a blood pressure, a posture indicator, an exercise indicator, and/or an endogenous brain pressure.

At 304, operations include calculating an estimated fluid production rate based on the one or more biometric indicators associated with the patient.

At 306, operations include calculating a desired value based on the estimated fluid production rate. In some examples, the desired value includes a desired temperature.

At 308, operations include communicating with a valve to regulate fluid flowing therethrough based on the desired value. The valve can correspond to the valve 100, the valve 100′, and the valve 100″ described with respect to FIG. 1A, FIG. 1B and FIG. 1C, the valve 202 described with respect to FIG. 2, or the like. As described herein, the valve is a solid state valve. In some examples, the fluid flowing through the valve includes biological fluids in organs or tissues, such as cerebrospinal fluid (CSF) in brain tissue, blood in the circulatory system, and so on. The valve includes a housing, a tubing, a regulating element, an actuator, a sensor, a control component, and a power unit.

Referring to FIG. 3B, at 310, operations include receiving, by the control component of the valve, the desired value from the data processing device.

At 312, operations include controlling, by the control component of the valve, the actuator to actuate the regulating element. In some examples, the regulating element includes wax and at least one additive. In some examples, the actuator includes at least one of a temperature actuator, a mechanical actuator, an electro mechanical actuator. In implementations, since the valve can be implanted into a brain of a patient, it is preferable that the actuator is magnetic resonance imaging (MRI) compatible such that when the patient goes through an MRI procedure, the actuator would not interfere with the procedure. In some examples, the actuator can manipulate the regulating element to regulate resistance to the fluid flowing through the tubing. In some examples, the resistance is independent of intracranial pressure associated with the patient.

At 314, operations include sensing, by the sensor, a parameter associated with the regulating element. In some examples, the parameter associated with the regulating element is temperature associated with the regulating element.

At 316, operations include determining, by the control component of the valve, that the parameter associated with the regulating element is within a threshold range of the desired value.

At 318, operations include stopping, by the control component of the valve, the actuator upon determining that the parameter associated with the regulating element is within a threshold range of the desired value.

### (IV) Materials of Regulating Element Embodiments

As described herein, the reregulating element includes materials that are suitable to squeeze and/or release the tubing to regulate resistance to the fluid flowing through the tubing. For example, the regulating element includes wax and at least one additive (such as graphite and the like). These additives can improve thermal conductivity of the wax. They may not have a noticeable impact on the melting temperature of the wax. Table 1 shows examples of additives, and their proportion by mass.

TABLE 1

| | Minimum proportion | Maximum proportion |
|---|---|---|
| Additive Name | by mass (%) | by mass (%) |
| Graphite | 1 | 15 |
| Aluminum oxide | 1 | 15 |
| Iron oxide (Fe₃O₄) | 0.5 | 5 |
| Aluminum nitride | 0.5 | 5 |
| Atomized copper | 1 | 15 |

Note that the examples shown above are illustrative rather than limiting. There can be other types of additives. Moreover, the proportions of additives can vary based on actual needs.

### (V) Algorithm for Controlling Valve Embodiments

As described herein, systems and method described herein can match CSF outflow to CSF inflow (CSF production). A shunt with a valve according to this disclosure can regulate the CSF outflow for an individual patient and live over time. Activity-based changes in pressure (such as a sneezing, coughing, straining from constipation, exercising, and standing up) will not impact the outflow rate of the valve. Systems and method described herein can improve patients' quality of life in the short term by stopping drainage induced headache and in the long term by reducing the shunt failure rate. Algorithm for estimating CSF production can be used to control the valve in accordance with this disclosure. In some examples, the algorithm for estimating CSF production can include the following: Cerebral blood flow→CSF inflow (production).

The cerebral blood flow can be used to estimate CSF inflow (production). At baseline, Baghbani assumption is used that cerebral blood flow is to CSF production just as electrical voltage is to voltage with resistors. CSF production is cerebral blood flow with resistance due to capillaries, tissue compliance, arterial resistance, venous resistance, resistance from arachnoid, granulations.

CSF production rate is a function of mean arterial pressure (MAP). CSF production rate and intracranial pressure (ICP) are directly dependent on blood pressure. However, normal patients and hydrocephalic patients have different relationship of the CSF production rate with the blood pressure.

$$CSF {inflow}_{n o rmal} -> CSF {inflow}_{hydrocephalus}$$

The normal CSF inflow rate is used to estimate the hydrocephalous CSF inflow rate. CSF production (based on calculated and recorded resistances) is used as input to modify based on hydrocephalic bulk flow rate, flow amplitude, ICP in mild, moderate, severe hydrocephalus (Silverberg et al., J Neurosurg. 97 (6): 1271-1275, 2002, Juhler et al., Childs Nervous System, 16 (7), 446-9, discussion 450, 2020).

$$CSF {inflow}_{hydrocephalus} -> CSF {outflow}_{hydrocephalus}$$

The CSF inflow can be used to determine the CSF outflow of the valve.

As an example, an average CSF production rate can be 0.3-0.6 milliliter (mL)/min for normal people, 0.4±0.13 mL/min for acute hydrocephalus to moderate/mild hydrocephalus patients, and 0.25±0.08 mL/min for chronic hydrocephalus to severe hydrocephalus patients. (Silverberg et al., J Neurosurg. 97 (6): 1271-1275, 2002).

Based on nine studies, reviewed by M Czosnyka and M Juhler et al., some intracranial pressures are: (1) supine position with a mean ICP of 8.6 mmHg (standard deviation (SD) 4.7, reference interval 0.9 to 16.3 mmHg), (2) upright position with a mean ICP of 1.0 mmHg (SD 4.3, reference interval 5.9 to 8.3 mmHg), (3) continuous daytime measurement with a mean ICP of 0.1 mmHg (SD 7.4, reference interval 12.0 to 12.2 mmHg), and (4) continuous nighttime measurement with a mean ICP of 6.3 mmHg (SD 13.3, reference interval 15.8 to 28.2).

$$CSF {outflow}_{hydrocephalus} -> Temperature$$

The CSF outflow of the valve can be used to calculate the desired temperature of the regulating element.

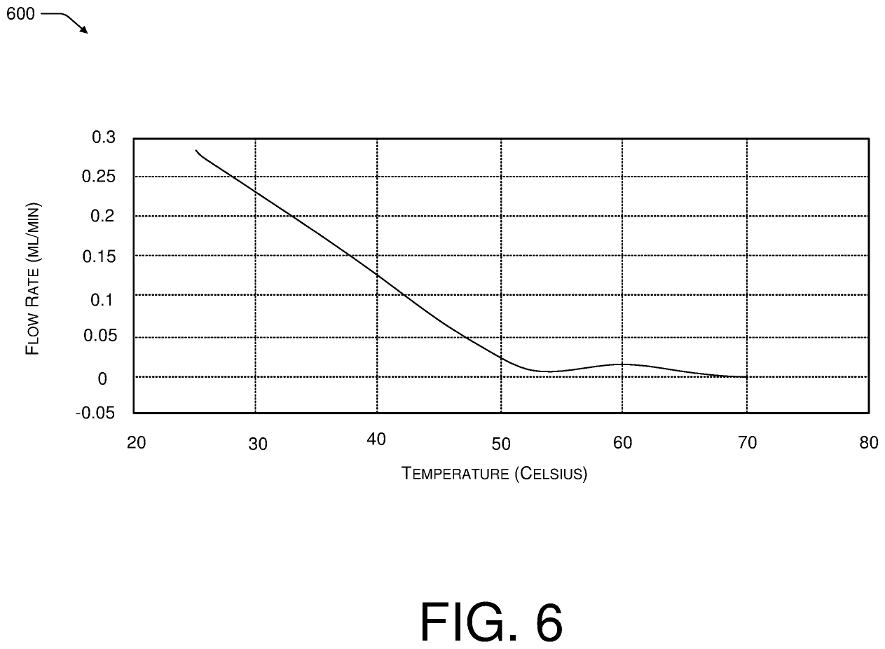

FIG. 6 illustrates a graph 600 of the relationship of the flow rate versus the temperature of the regulating element. In the graph 600, the horizontal axis represents the temperature in Celsius, and the vertical axis represents the flow rate in mL/min.

### (VI) Kits

Also provided are kits useful for treating hydrocephalus patients. An example of the kit includes one or more of: a wearable device, a shunt valve which can be sterile and vacuum sealed, and a lithium battery pack which is sterile, vacuum sealed.

More generally, kits can include instructions, for example written instructions, on how to use the material(s) therein. Material(s) can be, for example, any substance, composition, polynucleotide, solution, etc., herein or in any patent, patent application publication, reference, or article that is incorporated by reference.

A kit can include a device as described herein, and optionally additional components such as buffers, reagents, and instructions for carrying out the methods described herein. The choice of buffers and reagents will depend on the particular application, e.g., setting of the assay (point-of-care, research, clinical), analyte(s) to be assayed, the detection moiety used, the detection system used, etc.

The kit can also include informational material, which can be descriptive, instructional, marketing, or other material that relates to the methods described herein and/or the use of the devices for the methods described herein. In embodiments, the informational material can include information about production of the device, physical properties of the device, date of expiration, batch or production site information, and so forth.

### (VII) Catheter Designs

Provided herein is an improved catheter that can be used to treat hydrocephalus patients. The shape of the catheter and/or the sizes of the plurality of holes are configured to regulate one or more parameters of the fluid flowing through the catheter holes and/or the catheter. In some examples, the one or more parameters include at least one of a flow velocity, a shear rate, or a shear stress of the fluid flowing through the catheter holes and the catheter.

### (1) Structure of the Catheter

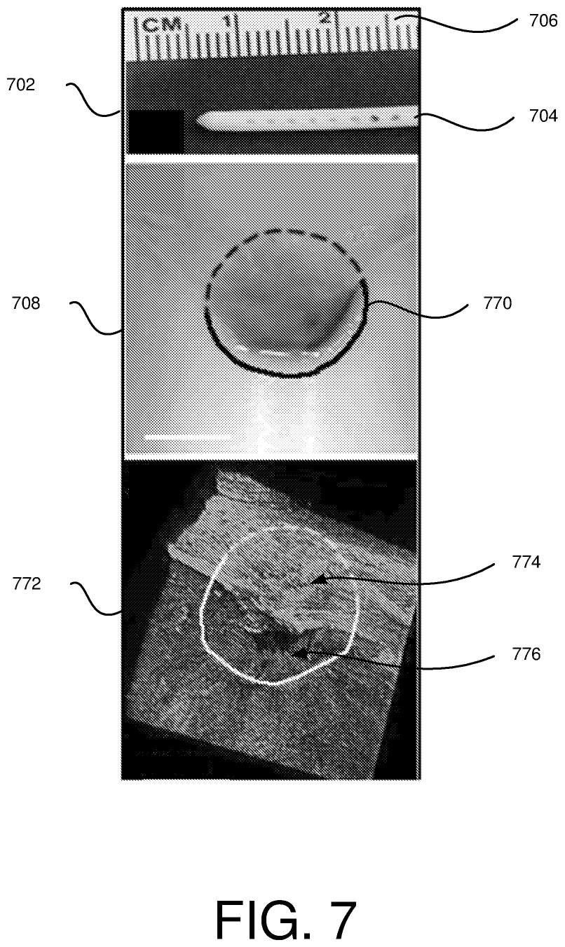

CSF shunt implantation is the most common treatment option for hydrocephalus, yet shunts are plagued by high failure rates: 40% in the first year, and 90% in the first 10 years. Hydrocephalus treatment fails most often because the outflow pathway created by the holes in the shunt's ventricular catheter becomes obstructed with tissue. Up until a few years ago, the most significant studies on shunt failure revealed that shunts most commonly harbor inflammatory glia, lymphocytic inflammation, and foreign body giant cells. Work shows that the tissue occluding shunts is predominately composed of astrocytes and macrophages, has only sparse microglia, has more activated cells on obstructed shunts than unobstructed, stain positive for proliferative markers, has reactivity that follows the flow, and predominately obstructs shunts as large tissue masses. FIG. 7 illustrates pictures of a catheter and blocked catheter holes. In FIG. 7, picture 702 shows an example catheter 704 along with a ruler 706. Picture 708 shows a hole 770 of the catheter being blocked. The scale bar=500 microns. Picture 772 shows that astrocytes 774 and macrophages 776 block the catheter hole as a large tissue mass.

Recent long-term in vivo data indicate that inflammatory astrocytes are ubiquitous on all shunts. Inflammatory astrocytes make up more than 21% of cells bound to obstructed shunts, and of the occluded masses blocking ventricular catheter holes, a vast majority of the cells are astrocytes, and their number and reactivity peak on failed shunts. Data suggests that inflammatory astrocyte activation on shunts is correlated to a change in flow rate through the shunt holes and indirectly, the shear rate through these holes (Harris et al., Childs Nerv. Syst., 2011; doi.org/10.1007/s00381-011-1430-0; Harris et al., Exp. Neurol. 222:204-210, 2010; Harris et al., J. Biomed. Mater. Res. A 97:433-440, 2011; Harris et al., Fluids Barriers CNS. 2015, doi.org/10.1186/s12987-015-0023-9). Astrocytes have an increased attachment propensity in vitro with increasing flow-induced shear stress. Astrocyte markers have been observed in obstructive masses to be co-localized with proliferative markers, indicating that astrocytes are active on the shunt surface: they produce inflammatory cytokine IL-6 and proliferate.

Most of the CSF volume flows through the proximal holes of the shunt's ventricular catheter, i.e., holes located furthest from the tip of the shunt with less resistance to flow. Computational fluid dynamics simulations have shown that in CSF shunts, the wall shear stress at the proximal holes is greater than 0.5 dyne/cm² (Harris et al., Childs Nerv. Syst., 2011; doi.org/10.1007/s00381-011-1430-0; Lin et al., J. Neurosurg. 99:426-431, 2003; Lee et al., J. R. Soc. Interface 17, 20190884, 2020). This fact increases the shear stress at the proximal segment and is a key driver of a dense glial scar formation around devices causing failure via obstruction (Lin et al., J. Neurosurg. 99:426-431, 2003; Giménez et al., Philos. Trans. A Math. Phys. Eng. Sci. 375:20160294, 2017; Marimuthu et al., Anal. Biochem. 437:161-163, 2013).

CSF shunts removed for obstruction show occlusions to occur most often at the proximal holes (holes located furthest from the tip) with the highest flow (Kestle et al., Pediatr. Neurosurg. 33:230-236, 2000). These observations led to a suggestion that shunt geometry with a more uniform flow rate distribution among the shunt's inlet holes would reduce the obstruction occurring at the critical proximal inlet holes, thereby reducing shunt failure rates. As an example of an existing shunt catheter, the Rivulet® (Medtronic Neurosurgery) shunt was developed with a design consisting of decreasing hole diameters from the distal to proximal end (Lin et al., J. Neurosurg. 99:426-431, 2003). However, shear stress will be higher in the proximal holes of these shunts. Based on the fluid shear stress equation of T=μ du/dy, where u is dynamic viscosity, and du/dy is the gradient of velocity in the direction perpendicular to the flow, since the gradient velocity of the decreasing hole diameters is higher, shear stress will be higher for the proximal holes. Based on our hypothesis and other reports of the correlation between increased shear stress along the shunt/CSF interface to result in increased occlusion (Harris et al., Childs Nerv. Syst., 2011; doi.org/10.1007/s00381-011-1430-0; Harris et al., Exp. Neurol. 222:204-210, 2010; Harris et al., J. Biomed. Mater. Res. A 97:433-440, 2011; Galarza et al., Child′ Nerv. Syst. 34:267-276, 2018; Weisenberg et al., J. Neurosurg. 2017, doi.org/10.3171/2017.5.JNS161882), it may be necessary to improve shunt design to decrease shear stress through all its holes or at best the proximal holes.

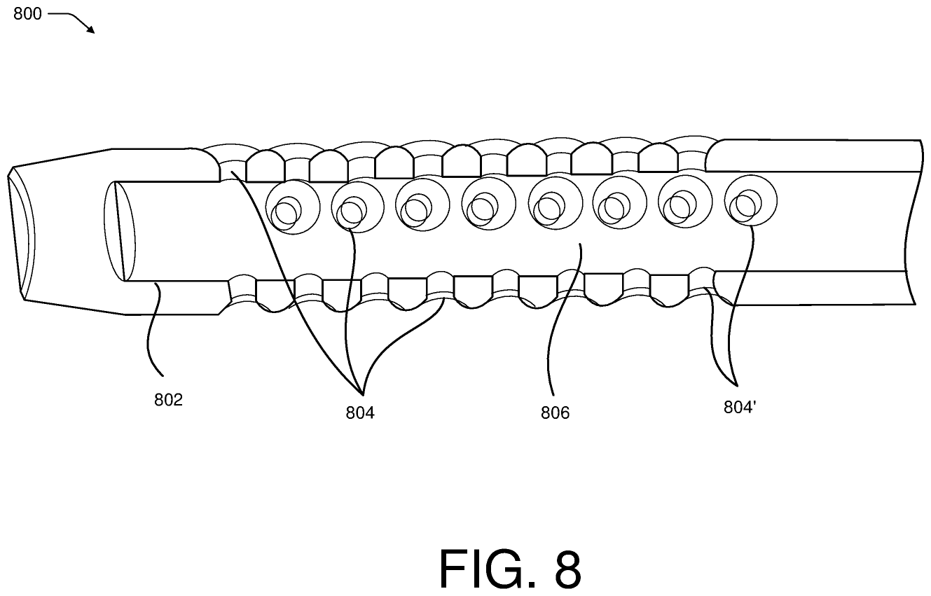

In implementations, hydrocephalus shunts come in various shapes and designs. FIG. 8 illustrates an example conventional catheter 800. Referring to FIG. 8, the catheter 800 includes a tip end 802, multiple holes 804, and a lumen 806. In this example, the catheter 800 is cylindrical, and the radius thereof is the same throughout the catheter 800. The catheter 800 includes multiple holes that allow fluid to flow therethrough into the lumen.

Among the multiple holes, the quantity of fluid increases as the fluid flows closer to the holes far from the tip end 802 (such as the holes 804′), which means the velocity of the fluid needs to increase. Increase in fluid velocity results in a reduction in pressure. A pressure gradient is created in the lumen 804 as the result of the flow pattern inside the lumen 804 of the catheter 800. This results in preferential flow through the holes with the highest fluid velocity (the lowest pressure). That is, the holes that are farthest from the tip end 802 (such as the holes 804′) can have more fluid flow therethrough than other holes. As such, the blockage can occur more often around the holes (such as the holes 804′) furthest from the catheter tip.

Therefore, there is a need to reduce the pressure gradient by maintaining a constant low velocity inside the lumen which can result in flow through all the catheter holes and can introduce additional pathways for fluid to go through. This can be done by optimizing the geometry of the catheter.

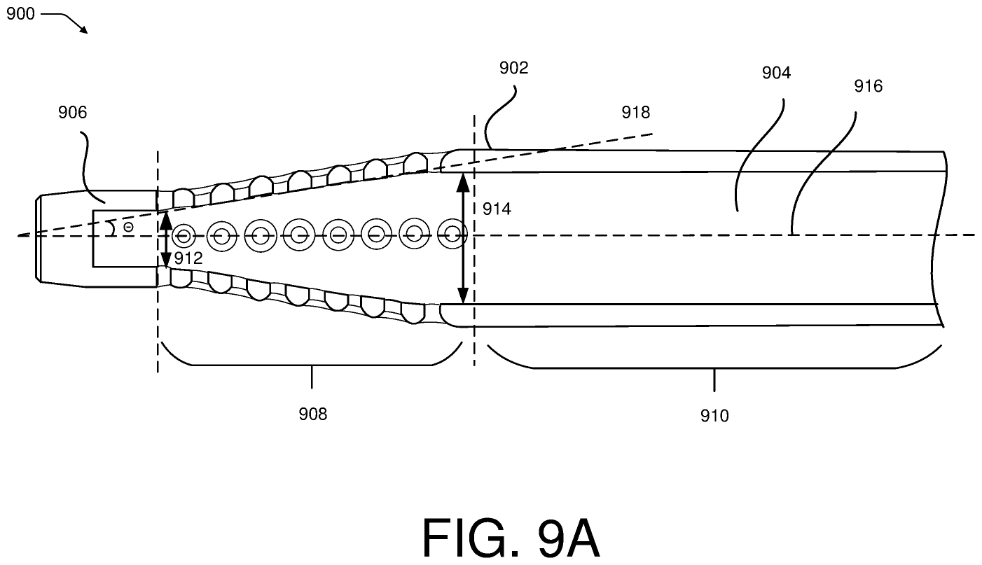

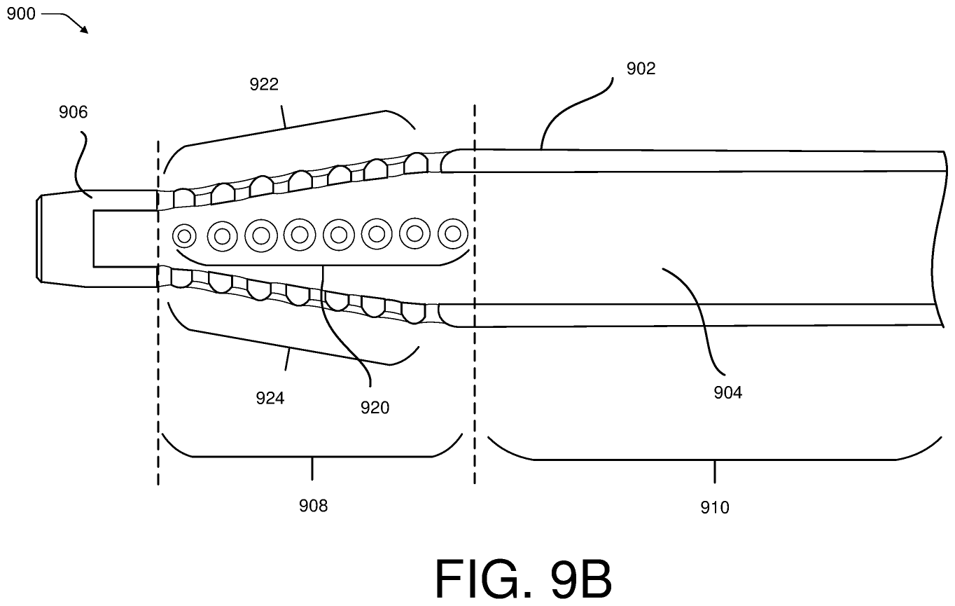

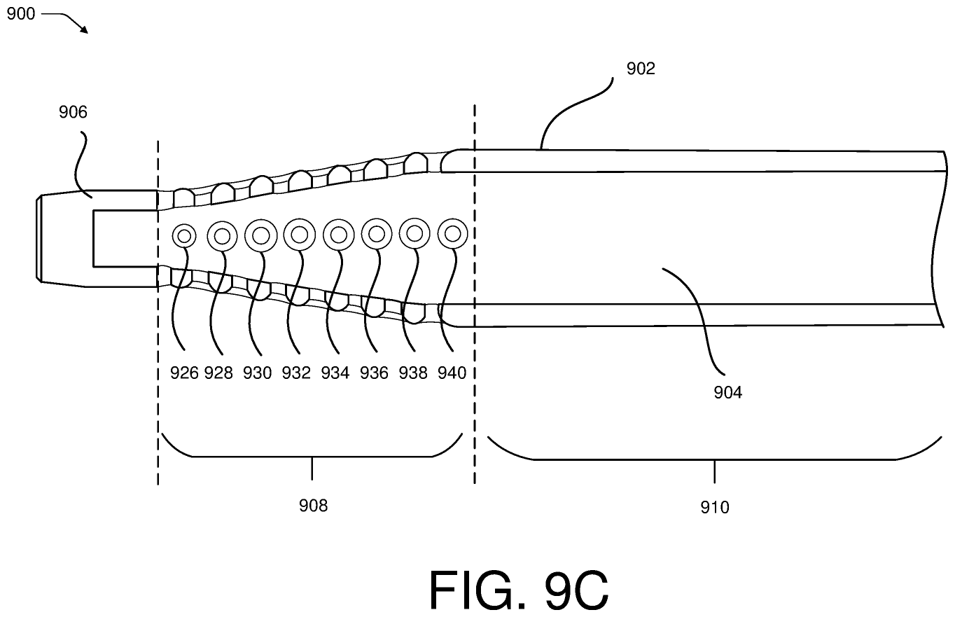

FIG. 9A, FIG. 9B, and FIG. 9C illustrate an example catheter 900 in accordance with this disclosure. As described herein, the catheter 900 can be used for shunting flow of biological fluids in organs or tissues including in vivo or in vitro such as in the context of cerebrospinal fluid in brain tissue, and blood in the circulatory system. For example, the catheter 900 can be implanted into a patient's brain ventricle to shunt CSF to treat hydrocephalous. In some examples, the catheter 900 can be used in vitro to study fluid flow. In some examples, the catheter 900 has an axisymmetric shape such as a cylindrical shape or the like. Moreover, the catheter 900 can have other shapes as long as the shape is suitable for shunting biological fluids.

Referring to FIG. 9A, the catheter 900 includes a sidewall 902. The sidewall 902 defines a lumen 904 of the catheter 900. The catheter 900 has a tip end 906, a first portion 908, and a second portion 910. The tip end 906 is a closed end. The first portion 908 is between and in fluid communication with the tip end 906 and the second portion 910. The first portion 908 can be configured to have a tapered shape, meaning that the diameter of the lumen 904 of the catheter expands gradually or step-wise from the tip end 906 toward the second portion 910. For example, the first portion 908 has a first diameter 912 near the tip end 906, and a second diameter 914 near the second portion 910. In some examples, the first diameter 912 is smaller than the second diameter. In some examples, the first diameter is between 1 millimeter (mm) and 4 mm. The second diameter 914 is between 2 mm and 5 mm. Examples of the first diameter 912 can be 1.5 mm, 1.6 mm, 1.7 mm, 3.5 mm, or the like. Examples of the second diameter 914 can be 2.45 mm, 2.8 mm, 3 mm, 4.5 mm, or the like.

As shown in FIG. 9A, the catheter 900 can have a central axis 916. The central axis 916 is a hypothetical line that passes through the center of the catheter longitudinally. In the first portion 908, the sidewall 902 can have a slope 918 towards the tip end 906. An angle θ of the slope 918 with respect to the central axis 916 can be between 0 degree and 90 degree. The second portion 910 has a constant diameter such as the second diameter 914.

In some examples, the ratio of the first diameter 912 to the second diameter 914 can be between 1:3 and 3:5. In some examples, instead of having a linear sloped sidewall, the first portion 908 can have a curved sidewall. In some examples, the first portion 908 can have a step-wise sidewall.

Referring to FIG. 9B, the catheter 900 can include one or more rows of holes on the sidewall 902 that allow biological fluid flow therethrough from an outside environment (such as a brain ventricle or the like) to the lumen 904 of the catheter 900. For example, in the first portion 908, the sidewall 902 includes a first row of holes 920, a second row of holes 922, a third row of holes 924, and a fourth row of holes (on the backside of the sidewall, not shown). In some examples, the shape of an individual hole can be round, square, or any other suitable shapes.

As an example, referring to FIG. 9C, the first row of holes 920 includes a first hole 926, a second hole 928, a third hole 930, a fourth hole 932, a fifth hole 394, a sixth hole 936, a seventh hole 938, and an eighth hole 940. Note that the number of holes is exemplary rather than limiting, and there can be other numbers of holes. In some examples, the first hole 926, the second hole 928, the third hole 930, the fourth hole 932, the fifth hole 934, the sixth hole 936, the seventh hole 938, and an eighth hole 940 can have the same diameter. In some examples, the first hole 926, the second hole 928, the third hole 930, the fourth hole 932, the fifth hole 934, the sixth hole 936, the seventh hole 938, and an eighth hole 940 can have different diameters. In some examples, the diameter of an individual hole can be between 200 μm and 700 μm. Examples of the diameter of an individual hole can include 600 μm, 750 μm, 450 μm, 460 μm, 275 μm, or the like.

In some examples, the first hole 926, the second hole 928, the third hole 930, the fourth hole 932, the fifth hole 934, the sixth hole 936, the seventh hole 938, and an eighth hole 940 can be placed with constant intervals. In some examples, the first hole 926, the second hole 928, the third hole 930, the fourth hole 932, the fifth hole 934, the sixth hole 936, the seventh hole 938, and an eighth hole 940 can be placed with various intervals. In some examples, the intervals between the holes can be between 0.5 mm to 4 mm. Examples of the intervals can include 3.7 mm, 3.45 mm, 1.1 mm, 0.8 mm, or the like.

Though FIG. 9A, FIG. 9B, and FIG. 9C shows that the multiple rows of holes are arranged in a linear manner, it should be understood that, the holes can be arranged in other manners. In some examples, the multiple rows of holes can be arranged in a spiral manner. In some examples, the holes can be arranged in a staggered manner.

### (2) Shunt System Including the Catheter

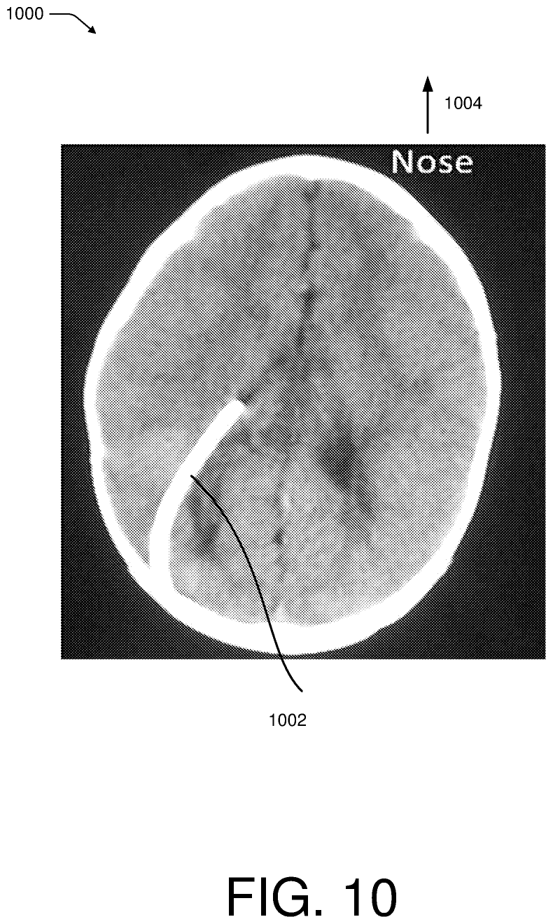

As described herein, the common treatment for hydrocephalus patients is CSF drainage by shunting. A shunt system can be implemented into a patient's brain by surgical insertion to treat hydrocephalous patients. FIG. 10 illustrates a horizontal head CT image 1000 showing a catheter tip implanted in a patient's brain. Arrow 1004 shows the direction of the patient's nose. Referring to FIG. 10, the image 1000 shows a catheter tip 1002 surrounded by tissue. While many factors such as infection and disconnection could lead to shunt obstruction and eventual failure, the statistics tell us that most shunts fail by becoming blocked with cells and tissues. As described above, shunt geometry with a more uniform flow rate distribution among the shunt's inlet holes would reduce the obstruction occurring at the critical proximal inlet holes, thereby reducing shunt failure rates.

This disclosure further provides a shunt system including the catheter for shunting biological fluid flow as described above. Such a shunt system offers a more uniform flow rate distribution among the inlet holes of the catheter, and would reduce the obstruction occurring at the inlet holes, thereby reducing shunt failure rates. The shunt system is can improve patients' quality of life by reducing the shunt failure rate. In some instances, the system can also be used to conduct in vitro experiments, collect analytic data, validate shunt functions, etc.

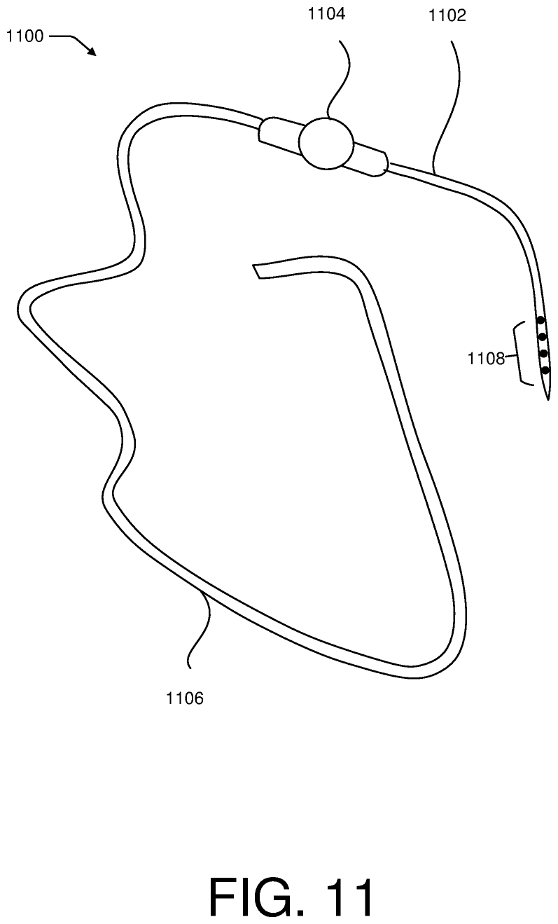

FIG. 11 illustrates an example shunt system 1100 in accordance with implementations of this disclosure. In some examples, the shunt system 1100 can be used in vitro to study fluid flow, or in vivo to treat or ameliorate symptoms of hydrocephalus. Referring to FIG. 11, the shunt system 1100 includes a ventricle catheter 1102, a valve 1104, and a distal catheter 1106. The valve 1104 is connected between the ventricular catheter 1102 and the distal catheter 1106. In some examples, the shunt system 1100 can be made of biologically compatible material such as polydimethylsiloxane (PDMS, silicone) or the like.

The ventricle catheter 1102 can be implemented into a patient's brain ventricle. The ventricle catheter 1102 includes multiple holes 1108 that allow biological fluid flowthrough into the ventricle catheter 1102. The ventricle catheter 1102 can be configured in the same way as the catheter 900 described with respect to FIG. 9A, FIG. 9B, and FIG. 9C.

The valve 1104 is configured to regulate the biological fluid (such as CSF) flowing therethrough. In some examples, the valve 1104 can be opened and closed. In some examples, the valve 1104 can regulate the flow rate of the biological fluid. In some examples, the valve 1104 can be a conventional valve. In some examples, the valve 1104 can be a solid state valve described throughout this application.

The distal catheter 1106 is configured to introduce the biological fluid to another part of the body, such as the abdomen through the peritoneum of the patient. As such, excess CSF of the hydrocephalous patient can be drained from the brain to another part of the body where CSF can be more easily absorbed.

Example shunts are composed of two polydimethylsiloxane (PDMS, silicone) shunt catheters connected by a pressure valve. One catheter remains in the ventricles, while the other is tunneled subcutaneously into the peritoneum or atrium.

### (3) Kits

Also provided are kits useful for treating hydrocephalus patients. An example of the kit includes one or more of: a ventricular catheter, a shunt valve, and a distal catheter, each of which is sterile, and vacuum sealed. The ventricular catheter can be configured in the same way as the catheter 900 described with respect to FIG. 9A, FIG. 9B, and FIG. 9C.

More generally, kits can include instructions, for example, written instructions, on how to use the material(s) therein. Material(s) can be, for example, any substance, composition, solution, etc., herein or in any patent, patent application publication, reference, or article that is incorporated by reference.

A kit can include a shunt system as described herein, and optionally additional components such as buffers, reagents, and instructions for carrying out the methods described herein. The choice of buffers and reagents will depend on the particular application, e.g., setting of the assay (point-of-care, research, clinical), analyte(s) to be assayed, the detection moiety used, the detection system used, etc.

The kit can also include informational material, which can be descriptive, instructional, marketing, or other material that relates to the methods described herein and/or the use of the devices for the methods described herein. In embodiments, the informational material can include information about the production of the device, physical properties of the device, date of expiration, batch or production site information, and so forth.

### (4) Simulation Results

As described herein, catheters and systems including the same were developed to reduce the pressure gradient by maintaining a constant low velocity inside the catheter. Such a system could facilitate flow through all the catheter holes and introduce additional pathways for fluid to go through. The shunt geometry was designed to facilitate a more uniform flow rate distribution among the shunt's inlet holes which would reduce the obstruction at the inlet holes, thereby reducing shunt failure rates. To validate whether the techniques described herein could work as expected, simulations were conducted, and data were collected.

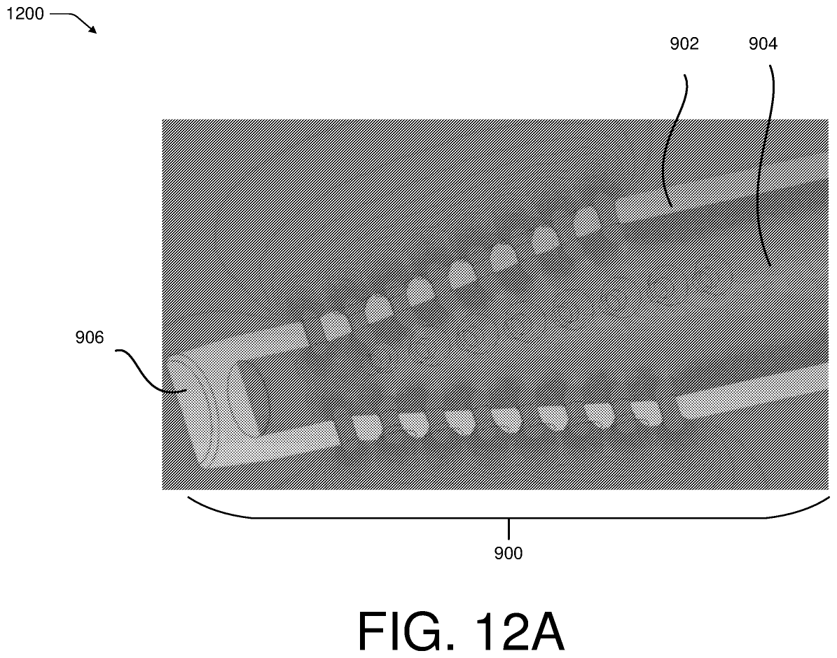

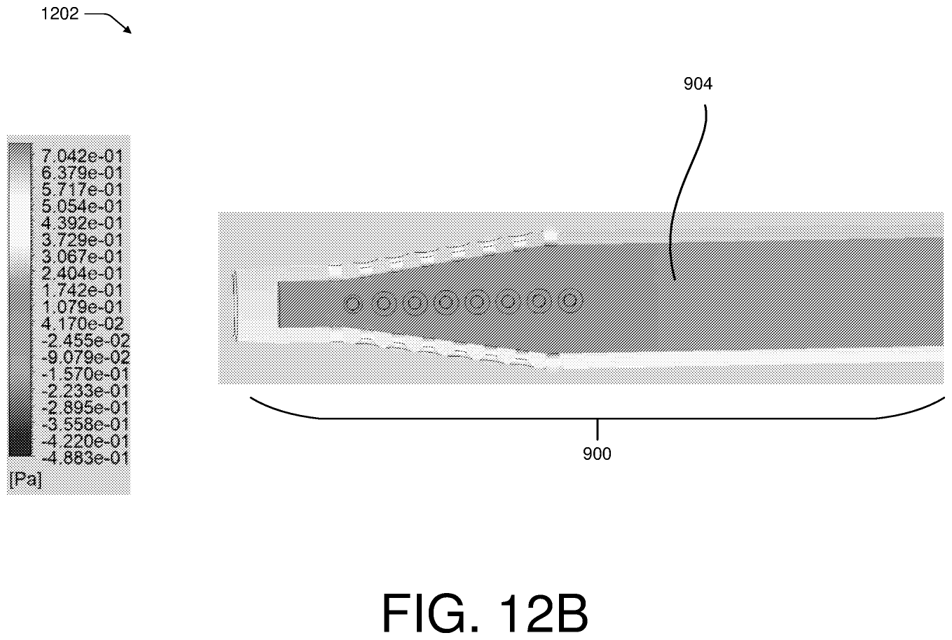

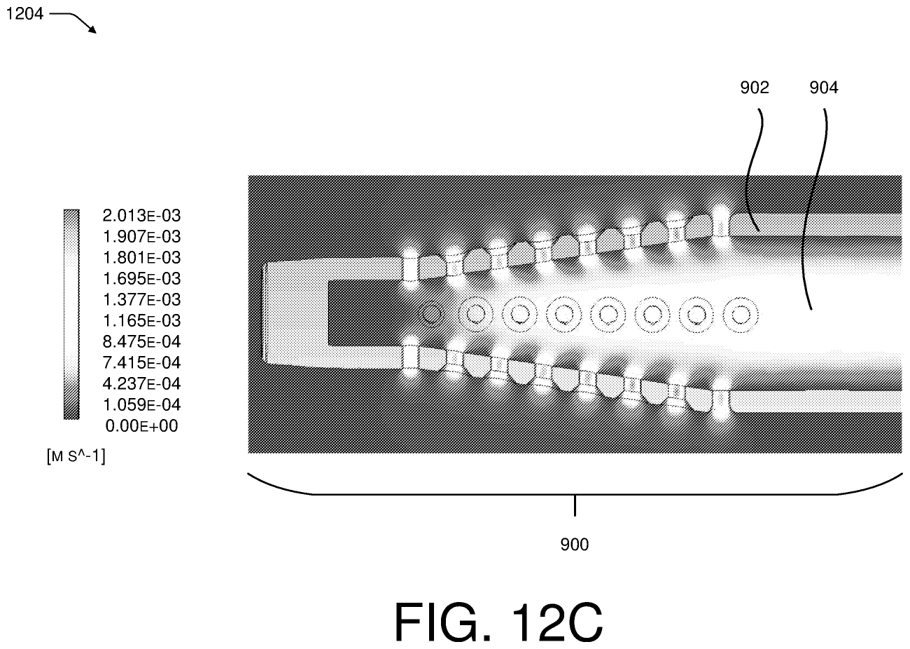

FIG. 12A shows a simulation result 1200 of the hydrodynamics of flow through the catheter 900 in accordance with implementations of this disclosure. In FIG. 12A, the greyscale indicated the velocity of the fluid. The darker the color was, the lower the velocity was. Note that the simulation result was schematic rather than limiting. There can be other manners to analyze the characteristic of the catheter 900. Referring to FIG. 12A, the velocity in the lumen 904 of the catheter 900 was substantially constant except for areas around the sidewall 906. As a result, the pressure gradient inside the lumen 904 were reduced by maintaining a substantially constant low velocity inside the lumen 904. Moreover, the fluid flew through most of the catheter holes rather than a few catheter holes that are farthest from the tip end 902. Therefore, it can be seen that the catheter 900 achieved a more uniform flow rate distribution among the shunt's inlet holes which would reduce the obstruction at the inlet holes. As such, shunt failure rates can be reduced.

FIG. 12B illustrates a simulation result 1202 of the pressure contours inside the catheter 900 in accordance with implementations of this disclosure. Referring to FIG. 12B, the pressure in the lumen 904 of the catheter 900 was substantially constant. In other words, the pressure gradient inside the lumen 904 decreased.

FIG. 12C illustrates a simulation result 1204 of the velocity contours inside the catheter 900 in accordance with implementations of this disclosure. Referring to FIG. 12C, the velocity in the lumen 904 of the catheter 900 was substantially constant except for areas around the sidewall 902. Moreover, the fluid flew through most of the catheter holes rather than a few catheter holes. Therefore, it can be seen that the catheter 900 achieved a more velocity distribution among the inlet holes which would reduce the obstruction at the inlet holes. As such, shunt failure rates can be reduced.

In some examples, the simulation was conducted using commercial software such as ANSYS, COMSOL, or the like.

The Example(s) and Exemplary Clauses below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (VIII) Structure and Function of Implantable Valve Embodiments

As described above, CSF drainage by shunting is a common treatment for hydrocephalus patients, and can be implemented with a shunt. In some cases, hydrocephalus patients can experience a high failure rate of shunts which can lead to diminished quality of life and suffering from long-term neurologic deficits. This disclosure provides an implantable valve with an active outflow regulator. The implantable valve according to embodiments of this disclosure is capable of controlling the CSF outflow by regulating the resistance of the tubing, which regulation is independent of the intracranial pressure and controlled by an external regulatory component. Tissue contact can be attenuated with the valve that minimizes over-drainage by matching the CSF outflow to the physiologic CSF production. This implantable valve offers protection against activity-based changes in pressure, and thus can improve a patient's quality of life in the short term by preventing over-drainage induced headaches and in the long term by reducing the shunt failure rate.

Moreover, other applications outside of hydrocephalus are also envisioned. For example, the valves provided by this disclosure, including those that are regulated by an external actuating component, can be used downstream from the proximal (ventricular) tip of a ventriculoperitoneal shunt, a ventriculoatrial shunt, a lumbar peritoneal shunt, or the like.

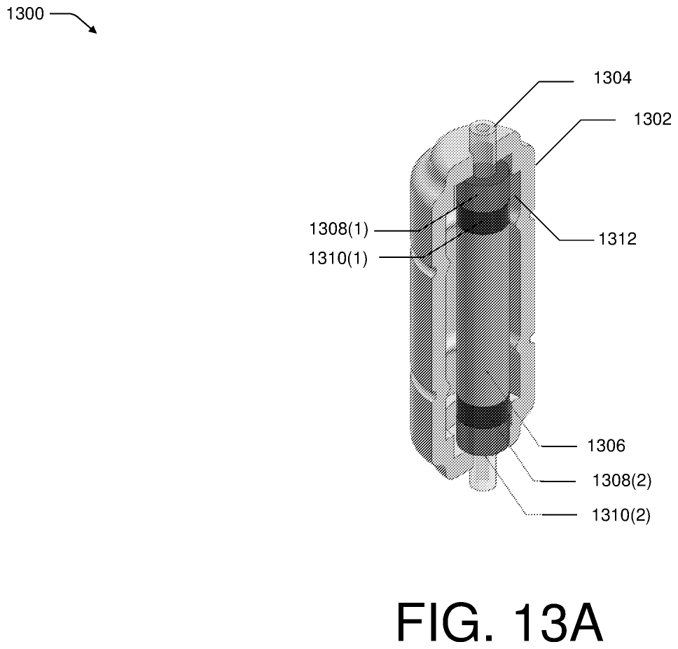

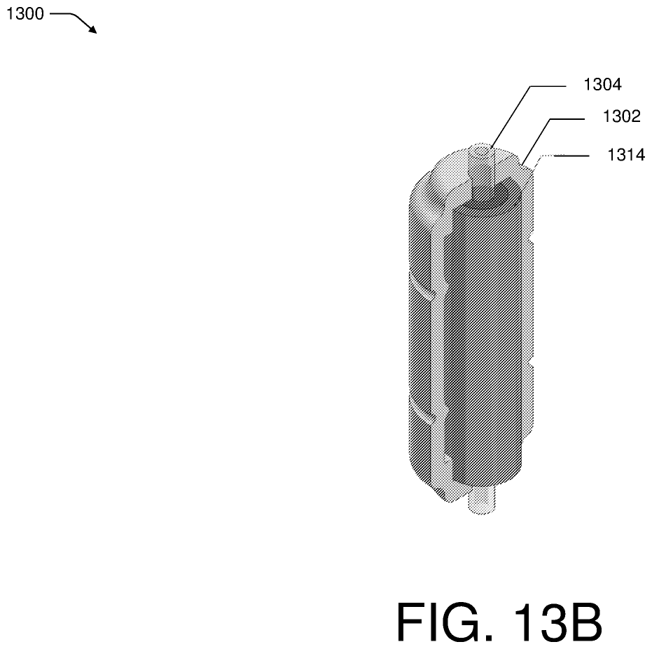

Also envisioned are embodiments where the system (for instance, including an implantable valve and an external actuating component) is used to deliver a fluid to a subject's body, rather than regulating removal of a fluid. In some instances, an implantable valve, including one regulated by an external actuating component, can be used to deliver drugs to a subject-such as insulin, antibiotics, or other drugs to the bloodstream, the brain, the spinal column, interperitoneally, and so forth. In some examples, when the valve is used for delivering drugs, it is used with a reservoir and a y or T connector before or after the valve. The catheter optionally may be surface coated or impregnated for chemical release. FIG. 13A and FIG. 13B illustrate an implantable valve 1300 for regulating fluid flow in accordance with implementations of this disclosure. The implantable valve 1300 is suitable to be implanted into a patient's body. In some examples, the implantable valve 1300 is a solid state valve. The solid state valve can refer to an implantable valve using materials that are not fluid but retain the solid state. In some examples, the fluid flowing through the implantable valve 1300 includes biological fluids in organs or tissues, such as cerebrospinal fluid (CSF) in brain tissue, blood in the circulatory system, and so on. In some examples, the implantable valve 1300 can be a part of a shunt system for treating hydrocephalous.

Referring to FIG. 13A, the implantable valve 1300 includes a housing 1302, a tubing 1304, a regulating element 1306, gaskets 1308(1) and 1308(2), sealant components 1310(1) and 1310(2), and a sensor (not shown). As described herein, the structure of the implantable valve 1300 is exemplary, and the implantable valve 1300 can include fewer or more components. In some examples, the gaskets 1308(1) and 1308(2) and the sealant components 1310(1) and 1310(2) can be omitted in the implantable valve 1300.

The housing 1302 is configured to accommodate the tubing 1304, the regulating element 1306, the gaskets 1308(1) and 1308(2), the sealant components 1310(1) and 1310(2), and the sensor. In some examples, an air insulating layer 1312 exists between the housing and inner components (e.g., the tubing 1304, the regulating element 1306, the gaskets 1308(1) and 1308(2), the sealant components 1310(1) and 1310(2), and the sensor), so as to reduce heat transmission between the inner components to the housing 1302.

In some examples, the housing 1302 includes at least one insulating material. Examples of the insulating material include insulating ceramic, polyethylene, polypropylene, or the like. In some examples, since the implantable valve 1300 can be implanted into the patient's brain, and the implantable valve 1300 may generate heat in use, the insulating material can inhibit the heat conduction from the implantable valve 1300 to the surrounding environment (such as brain ventricles). In some examples, the internal temperature needed for the implantable valve 1300 closure is 70° C. In some examples, the operating temperature for the implantable valve 1300 can be 50-70° C. In some examples, the operating temperature for the implantable valve can be 90-110° C. For examples, polyethylene which has a relatively low thermally conductivity of 0.33-0.5 W/m K can be used as the insulating material. As another example, polypropylene which has a relatively low thermally conductivity of 0.131 W/m K can be used as the insulating material.

In some examples, the housing 1302 can further include a heat sink (not shown), configured to dissipate away the heat generated by the implantable valve 1300, thereby decreasing of the temperature of the implantable valve 1300. Examples of heat sinks can include passive heat sinks, active heat sinks, metal heat sinks (such as aluminum heat sinks, copper heat sinks, or the like), heat sinks with coolant, or the like. Passive heat sinks are those that do not rely on forced air flow (fans). Active heat sinks have a powered device such as a fan or blower in close proximity to the heat exchanger surface.

The tubing 1304 is configured to allow fluid flow therethrough. In some examples, biological fluid can flow through the tubing 1304 such as cerebrospinal fluid (CSF) in brain tissue, blood in the circulatory system, and so on. In some examples, the fluid flow through the tubing can include drugs such as insulin, antibiotics, or other drugs.

The regulating element 1306 is arranged proximate to the tubing 1304. In some examples, the regulating element 1306 includes materials that are suitable to squeeze and/or release the tubing to regulate resistance to the fluid flowing through the tubing 1304. The resistance is independent of intracranial pressure associated with a patient. For example, the regulating element 1306 includes wax and at least one additive. Examples of the additive include graphite, ferromagnetic material (e.g., iron oxide, or the like), and the like. The graphite can be added to the wax to adjust the melting temperature of the regulating element 1306. When there is an applied electromagnetic field, the ferromagnetic material can generate an induced electromagnetic field to generate heat to raise the temperature of the regulating element 1306. In some examples, the ferromagnetic material can be used to form powders, beads, irregular pieces, or the like to be added to the wax. In some examples, the ferromagnetic material can take the form of electroplating. In implementations, the mass of the ferromagnetic material can be limited such that the ferromagnetic material would produce little impact on MRI (e.g., small artifacts without long-term manipulation of the MRI results). As an example, for wax with a volume of 2 mL, the mass of the ferromagnetic material add can be 0.1 to 0.6 gram (g).

The gaskets 1308(1) and 1308(2) and sealant components 1310(1) and 1310(2) are arranged between the regulating element 1306 and the housing 1302 to seal the gaps between the regulating element 1306 and the housing 1302. In some examples, the gaskets 1308(1) and 1308(2) can be made of rubber, silicone, neoprene, polytetrafluoroethylene (known as PTFE or Teflon), a plastic polymer (such as polychlorotrifluoroethylene), and the like. In some examples, sealant components 1310(1) and 1310(2) can be made of rubbers, Poly Tetra Fluoro Ethylene (PTFE), resins, or the like.

The sensor (not shown) is configured to sense the parameter associated with regulating element 1306. In some examples, the sensor includes a temperature sensor. In some instances, the sensor is arranged proximate to the regulating element 1306. Note that other types of sensors can be used to detect parameters associated with the fluid flowing through the implantable valve 1300, such as a flow rate sensor, a pressure sensor, or the like. In some instances, a sensor is arranged proximate to the tubing 1304 or inside the tubing 1304.

As shown in FIG. 13B, in some examples, the implantable valve 1300 further includes a heating element 1314 arranged proximate to the regulating element 1306. In this example, the heating element 1314 is in the form of a cylindrical shell. Additionally, the heating element 1314 can take other forms such as a mesh, a ring, a stick, a strap, or the like. The heating element 1314 can be made of a metal (such as iron, cobalt, nickel, or the like) or mixture of metals (such as aluminum, nickel, or the like), alloys (such as stainless steel), or other suitable materials. When there is an applied electromagnetic field, the heating element 1314 can generate an induced electromagnetic field, which can increase the temperature of the heating element 1314. The heating element 1314 can heat the regulating element 1306. In implementations, the mass of the heating element 1314 can be limited such that the heating element 1314 would produce little impact on MRI (e.g., small artifacts without long-term manipulation of the MRI results).

### (IX) Overview of the System Embodiments with Implantable Valve and External Actuating Component

This disclosure also provides a system including an implantable valve and an External actuating component for regulating biological fluid flow. In some instances, the system can be used to treat hydrocephalous patients. At least a part of the system can be implanted into a patient's brain. Such a system offers protection against activity-based changes in pressure, and is specifically engineered to improve patients' quality of life in the short term by preventing over-drainage induced headaches and in the long term by reducing the shunt failure rate. In some instances, the system can also be used to conduct in vitro experiments, to collect analytic data, to validate shunt valve functions, etc. In some embodiments, the system can be used to deliver fluids to a subject's body, for instance drugs such as insulin, antibiotics, or other drugs. These may be delivered in a targeted manner governed by the placement of indwelling components of the system, for instance to the bloodstream, the brain, the spinal column, interperitoneally, and so forth. In some examples the system can also be referred to as a “drainage catheter system,” a “delivery catheter system,” “indwelling catheter”, “indwelling probe,” or the like

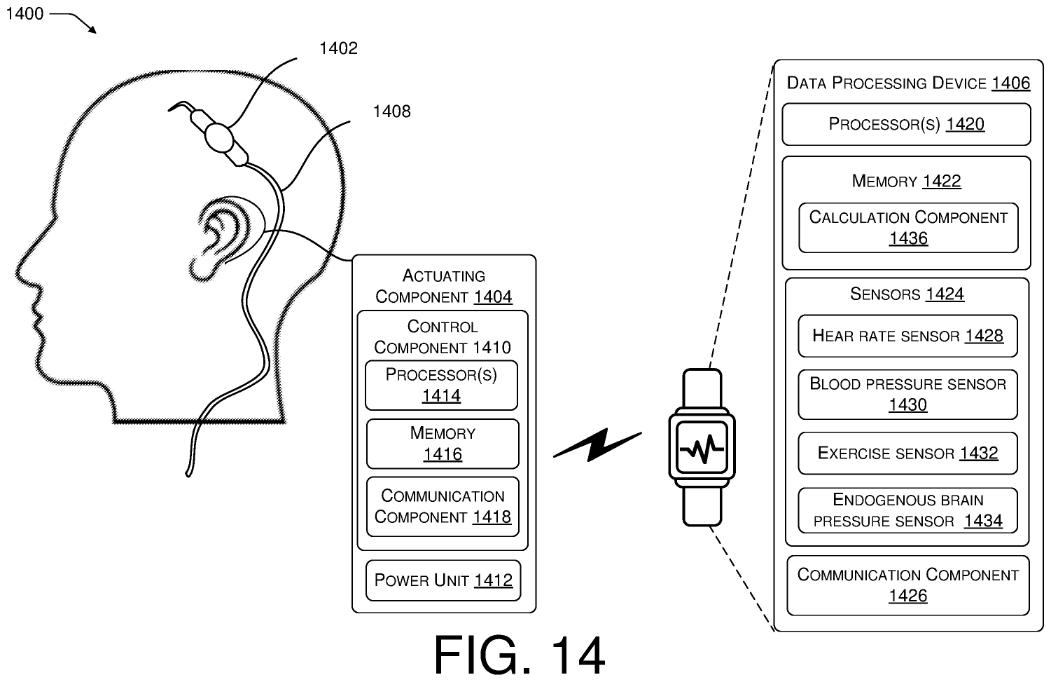

FIG. 14 illustrates an example system 1400 including an implantable valve 1402 and an actuating component 1404 in accordance with implementations of this disclosure. The implantable valve 1402 is suitable to be implanted into a patient's body for operation. The actuating component 1404 in some embodiments is suitable to be arranged outside the patient's body. Additionally, in some embodiments, the actuating component 1404 is suitable to be arranged inside the patient's body (that is, an internal or indwelling actuating component). Referring to FIG. 14, the system 1400 further includes a data processing device 1406 in communication with the actuating component 1404 wired or wirelessly via one or more networks. For example, the network(s) can include the Internet, a Mobile Telephone Network (MTN), Wi-Fi, a cellular network (e.g., 2G, 3G, 4G, 4G LTE, 5G, etc.), a mesh network, a Local Area Network (LAN), a Wide Area Network (WAN), a Virtual LAN (VLAN), a private network, short range wireless frequencies such as Bluetooth, and/or other various wired or wireless communication technologies.

The implantable valve 1402 for regulating fluid flow can be the same as the implantable valve 1300 as described with respect to FIG. 13A and FIG. 13B. In some examples, the implantable valve 1402 is a solid state valve. In some examples, the implantable valve 1402 is configured to regulate resistance to the fluid flowing therethrough.

The system 1400 further includes tubing 1408 is configured to allow fluid flow therethrough. In some examples, the implantable valve 1402 and a part of the tubing 1408 can be implanted into a patient's brain. In some examples, biological fluid can flow through the implantable valve 1402 and the tubing 1408 such as cerebrospinal fluid (CSF) in brain tissue, blood in the circulatory system, and so on. In some examples, the fluid flow through the implantable valve 1402 and the tubing 1408 can include drugs such as insulin, antibiotics, or other drugs.

The actuating component 1404 is configured to manipulate a parameter associated with a regulating element (not shown) in the implantable valve 1402. In some examples, the parameter associated with the regulating element can be the temperature associated with the regulating element. The actuating component 1404 is arranged outside a patient's body but proximate to the implantable valve 1402. In this example, the actuating component 1404 takes the form of an earpiece, which can be conveniently attached to the patient's ear. Note that the actuating component 1404 can take other forms of such as a necklace, a headband, a headset, a hairpin, a button, and the like, which can conveniently be attached to the patient's body. In some examples, the actuating component 1404 includes an electromagnetic generator (e.g., a coil, a magnetic solenoid, or the like), configured to apply an electromagnetic field to the implantable valve 1402. In some examples, a heating element (not shown) in the implantable valve 1402 can generate an induced electromagnetic field because of the electromagnetic field applied by the actuating component 1404. Additionally or alternatively, the ferromagnetic material included in the regulating element of the implantable valve 1402 can generate an induced electromagnetic field because of the electromagnetic field applied by the actuating component 1404. The induced electromagnetic field generated by the heating element and/or the ferromagnetic field can heat the regulating element of the implantable valve 1402, so as to regulate the fluid flow through the implantable valve 1402.

In implementations, since a part of the system 1400 (such as the implantable valve 1402 and a part of the tubing 1408) can be implanted into a brain of a patient, it is preferable that the system 1400 is magnetic resonance imaging (MRI) compatible such that when the patient goes through an MRI procedure, the system 1400 would not interfere with the MRI procedure. In this example, the actuating component 1404 can be conveniently removed from the patient's body when the patient is going through the MRI procedure, such that the system 1400 would not interfere with the MRI procedure.

The implantable valve 1402 includes a sensor (not shown), configured to sense the parameter associated with the regulating element. In some examples, the sensor includes a temperature sensor. In some instances, the sensor is arranged proximate to the regulating element. Note that other types of sensors can be used to detect parameters associated with the fluid flowing through the implantable valve 1402, such as a flow rate sensor, or a pressure sensor, or the like. In some instances, a sensor is arranged proximate to the tubing 1408 or inside the tubing 1408.

The actuating component 1404 further includes a control component 1410. The control component 1410 is configured to communicate with other devices and to control the actuating component 1404. In some examples, the control component 1410 can communicate with the other devices via network(s). For example, the control component 1410 can communicate with the data processing device (such as a wearable device) 1406 to receive data or instructions useful for controlling the actuating component 1404.

The control component 1410 can control the actuating component 1404 to generate the electromagnetic field to actuate the regulating element. For example, the control component 1410 can receive a desired value (such as a desired temperature) from the data processing device 1406. The sensor can sense the parameter (such as the temperature) associated with the regulating element. The control component 1410 can determine that the parameter (such as the temperature) associated with the regulating element is within a threshold range of the desired value (such as the desired temperature). The control component 1410 can stop the actuating component 1404 from generating the electromagnetic field upon determining that the parameter (such as the temperature) associated with the regulating element is within a threshold range of the desired value (such as the desired temperature). The threshold range can be set arbitrarily. As an example, the regulating element includes materials (such as wax and additives) that melt when the temperature increases and compresses the tubing 1408. As such, the fluid flow (such as CSF flow) through the tubing 1408 is reduced. The sensor can keep sensing the temperature associated with the regulating element. When the control component 1410 determines that the temperature associated with the regulating element is within a threshold range of the desired value (such as the desired temperature), the control component 1410 can stop the actuating component 1404 from generating the electromagnetic field to stop heating the regulating element 106.

The actuating component 1404 further includes a power unit 1412, configured to provide power for the actuating component 1404. In some instances, the power unit 1412 includes a Lithium battery. In some examples, the power unit 1412 can be recharged.

The control component 1410 includes one or more processors 1414, a memory 1416, and a communication component 1418. The processor(s) 1414 can be a single processing unit or a number of units, each of which could include multiple different processing units. The processor(s) 1414 can include a microprocessor, a microcomputer, a microcontroller, a digital signal processor, a central processing unit (CPU), a graphics processing unit (GPU), a security processor, etc. Alternatively, or in addition, some or all of the techniques described herein can be performed, at least in part, by one or more hardware logic components. For example, and without limitation, illustrative types of hardware logic components that can be used include a Field-Programmable Gate Array (FPGA), an Application-Specific Integrated Circuit (ASIC), an Application-Specific Standard Products (ASSP), a state machine, a Complex Programmable Logic Device (CPLD), pulse counters, resistor/coil readers, other logic circuitry, a system on chip (SoC), and/or any other devices that perform operations based on instructions. Among other capabilities, the processor(s) 1414 can be configured to fetch and execute computer-readable instructions stored in the memory.

The memory 1416 can include one or a combination of computer-readable media. As used herein, “computer-readable media” includes computer storage media and communication media. Computer storage media includes volatile and non-volatile, removable, and non-removable media implemented in any method or technology for storage of information, such as computer-readable instructions, data structures, program modules, or other data. Computer storage media includes, but is not limited to, Phase Change Memory (PRAM), Static Random-Access Memory (SRAM), Dynamic Random-Access Memory (DRAM), other types of Random-Access Memory (RAM), Read Only Memory (ROM), Electrically Erasable Programmable ROM (EEPROM), flash memory or other memory technology, Compact Disk ROM (CD-ROM), Digital Versatile Disks (DVD) or other optical storage, magnetic cassettes, magnetic tape, magnetic disk storage or other magnetic storage devices, or any other medium that can be used to store information for access by a computing device. In contrast, communication media includes computer-readable instructions, data structures, program modules, or other data in a modulated data signal, such as a carrier wave. As defined herein, computer storage media does not include communication media.

The memory 1416 can include an operating system configured to manage hardware and services for the benefit of other components and devices.

The communication component 1418 is configured to communicate with other devices (e.g., in the data processing device 1406, and so on) via network(s). For example, the communication component 1418 can perform compression, encryption, and/or formatting of the data received and/or generated by sensors. In some embodiments, the communication component 1418 can transmit data using one or more protocols or languages, such as an extensible markup language (XML), Modbus, HTTP, HTTPS, USB, etc.

The data processing device 1406 is configured to collect one or more biometric indicators associated with the patient. In some examples, the one or more biometric indicators associated with the patient include a heart rate, a blood pressure, a posture indicator, an exercise indicator, and/or an endogenous brain pressure. In some examples, the data processing device 1406 includes, but is not limited to, smart watches, smart accessories (e.g., smart rings, smart wristbands, smart pins, smart bracelets, or the like), body-mounted sensors, fitness trackers, smart clothing (e.g., smart shirt, smart pants, smart belt, or the like), smart headband, smart headsets, smart footwears, smart glasses, or the like.

The data processing device 1406 includes one or more processors 1420, a memory 1422, one or more sensors 1424, and a communication component 1426. The processor(s) 1420, the memory 1422, and the communication component 1426 can be implemented in the same manner as the processor(s) 1414, the memory 1416, and the communication component 1418.

The sensor(s) 1424 include a heart rate sensor 1428 configured to sense a heart rate associated with the patient, a blood pressure sensor 1430 configured to sense the blood pressure associated with the patient, an exercise sensor 1432 configured to sense an exercising status associated with the patient, endogenous brain pressure sensor 1434 (such as telemetric Intracranial pressure sensors) configure to sense endogenous brain pressure associated with the patient. The sensor(s) 1424 can further include other sensors suitable for detecting biometric indicators associated with the patient.

The data processing device 1406 is further configured to calculate an estimated fluid production rate based on the one or more biometric indicators associated with the patient. The memory 1422 includes a calculation component 1436 storing computer-readable instructions executable by one or more processors, that when executed by the one or more processors, cause the one or more processors to perform acts. For example, the calculation component 1436 can implement algorithms to calculate an estimated fluid production rate based on the one or more biometric indicators associated with the patient. The calculation component 1436 can implement algorithms to calculate a desired value based on the estimated fluid production rate. Additional details of algorithms are provided throughout this disclosure such as in section (VI).

The data processing device 1406 is further configured to send the desired value to the control component 1410 of the actuating component 1404. In this example, the desired value is the desired temperature of the regulating element. The control component 1410 can receive the desired value (such as the desired temperature of the regulating element) from the data processing device 1406 and use the desired value to control the actuating component 1404 to heat the regulating element to the desired temperature.

The techniques discussed above can be implemented in hardware, software, or a combination thereof. In the context of software, operations represent computer-executable instructions stored on one or more computer-readable storage media that, when executed by one or more processors, configure a device to perform the recited operations. Generally, computer-executable instructions include routines, programs, objects, components, data structures, and the like that perform particular functions or implement particular abstract data types.

### (X) “Earpiece” Designs

As described herein, a representative actuating component that is suitable to be arranged outside the patient's body can take various forms (such as an earpiece, a necklace, a headband, a headset, a hairpin, a button, and the like) that can be conveniently attached/detached to/from the patient's body. In implementations, since a part of the shunt system (such as the implantable valve and a part of the tubing) can be implanted into a brain of a patient, it is preferable that the shunt system is medical procedure (e.g., MRI) compatible such that when the patient goes through a medical procedure (such as an MRI), the shunt system would not interfere with that procedure. In implementations, the earpiece can be conveniently removed from the patient's body—for instance, when the patient is going through the MRI procedure, such that the shunt system would not interfere with the MRI procedure.

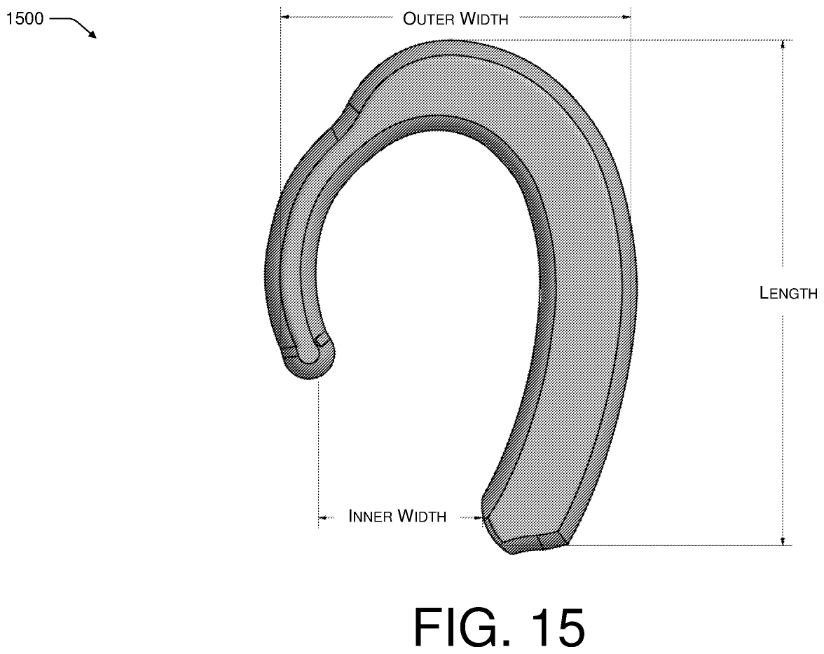

FIG. 15 illustrates an example earpiece 1500 in accordance with implementations of this disclosure. The earpiece 1500 can have an outer width, an inner width, and a length. As an example, the outer width is 155.74 mm; the inner width is 73 mm, and the length is 255.01 mm. Note that the dimensions, as well as the specific shape, of the earpiece 1500 are exemplary rather than limiting. The earpiece 1500 can have a shape and dimensions that are suitable for attaching/detaching to a human's ear.

In some examples, the earpiece 1500 includes an electromagnetic field generator (e.g., a coil, a magnetic solenoid, or the like), configured to apply an electromagnetic field to an implantable valve (such as the implantable valve 1300 described with respect to FIG. 13A and FIG. 13B). In some examples, a heating element in the implantable valve can generate an induced electromagnetic field because of the electromagnetic field applied by the earpiece 1500. Additionally or alternatively, ferromagnetic material included in a regulating element of the implantable valve can generate an induced electromagnetic field because of the electromagnetic field applied by the earpiece 1500. The induced electromagnetic field generated by the heating element and/or the ferromagnetic field can heat up the regulating element of the implantable valve, so as to regulate a resistance associated with the implantable valve to control the fluid flow through the implantable valve.

The earpiece 1500 further includes a control component (not shown). The control component is configured to communicate with other devices and to control the earpiece 1500. In some examples, the control component can communicate with the other devices via network(s) such as the Internet, a Mobile Telephone Network (MTN), Wi-Fi, a cellular network (e.g., 2G, 3G, 4G, 4G LTE, 5G, etc.), a mesh network, a Local Area Network (LAN), a Wide Area Network (WAN), a Virtual LAN (VLAN), a private network, short range wireless frequencies such as Bluetooth, and/or other various wired or wireless communication technologies. For example, earpiece 1500 can communicate with a data processing device (such as a wearable device) to receive data or instructions.

The control component is further configured to control the electromagnetic field generator to generate an electromagnetic field to heat the regulating element in the implantable valve. The function of the control component is the same as the function of the control component 1410 described with respect to FIG. 14. Details are not repeated here.

The earpiece 1500 further includes a power unit, configured to provide power for the earpiece 1500. In some instances, the power unit includes a Lithium battery. In some examples, the power unit can be recharged.

Though exemplified herein with an “earpiece” format, also contemplated are alternative configurations that maintain the external actuating component and power source of the system in close proximity with the regulating element of indwelling/implantable valve. In general, the external element(s) of the system are configured to be comfortably worn by the subject being treated, readily removable by the subject as needed, capable of holding the actuating component in functional proximity to the indwelling valve. For instance, a head band could be used, where the external actuating component is placed on the head proximal to the implantable valve assembly, or more particularly in proximity to the regulating element of the implantable valve.

### (XI) Characteristics of System Embodiments

As described herein, the characteristics of the system can be illustrated in various aspects. For example, the regulating element is used to control the opening/closing and the fluid flow of the implantable valve, including once it is implanted. For example, the regulating element can squeeze and/or release the tubing inside the valve to regulate resistance to the fluid flowing through the tubing. In embodiments, the regulating element includes wax and at least one additive. Examples of the additive include graphite, ferromagnetic material (e.g., iron oxide, or the like), and the like. The graphite can be added to the wax to adjust the melting temperature of the regulating element. The rate of heat exchange through the wax is influenced by additive composition and concentration.

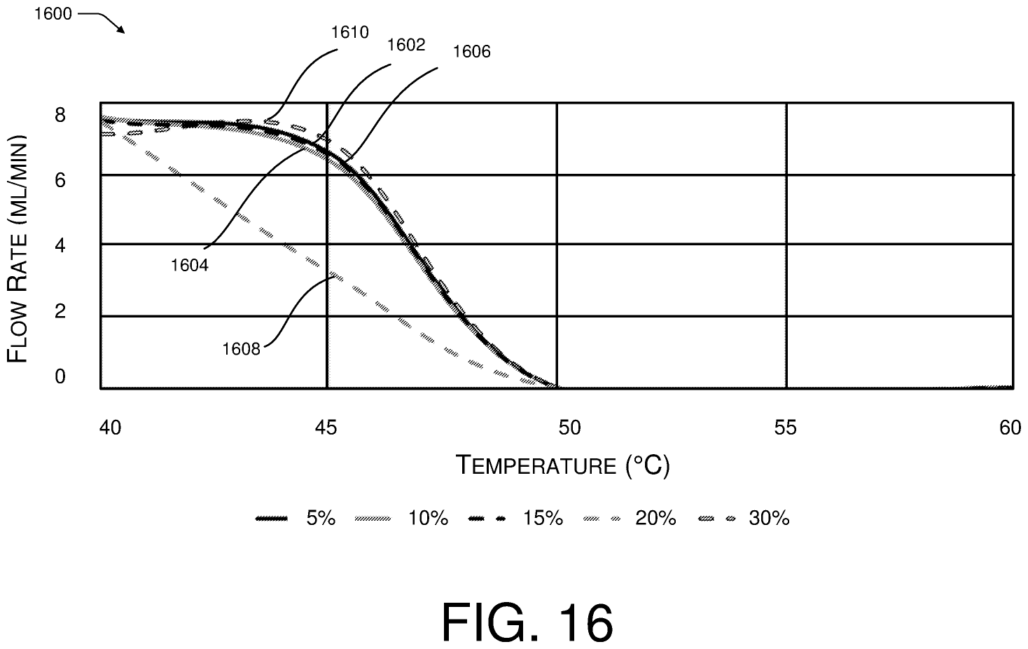

FIG. 16 illustrates a diagram 1600 showing curves of flow rates versus temperature where the regulating element has different additive (in this example, graphite) proportions. The curve 1602 shows the flow rate associated with the valve versus the temperature of the regulating element where the regulating element has an additive (in this example, graphite) proportion of 5%. The curve 1604 shows the flow rate associated with the valve versus the temperature of the regulating element where the regulating element has an additive (in this example, graphite) proportion of 10%. The curve 1606 shows the flow rate associated with the valve versus the temperature of the regulating element where the regulating element has an additive (in this example, graphite) proportion of 15%. The curve 1608 shows the flow rate associated with the valve versus the temperature of the regulating element where the regulating element has an additive (in this example, graphite) proportion of 20%. The curve 1610 shows the flow rate associated with the valve versus the temperature of the regulating element where the regulating element has an additive (in this example, graphite) proportion of 30%. Usually, the more additives in the regulating element, the faster the heat distributes across the regulating element. Thus, the faster compression to the tubing is applied by the regulating element, and thereby the flow rate of the fluid flow through the tubing is lower.

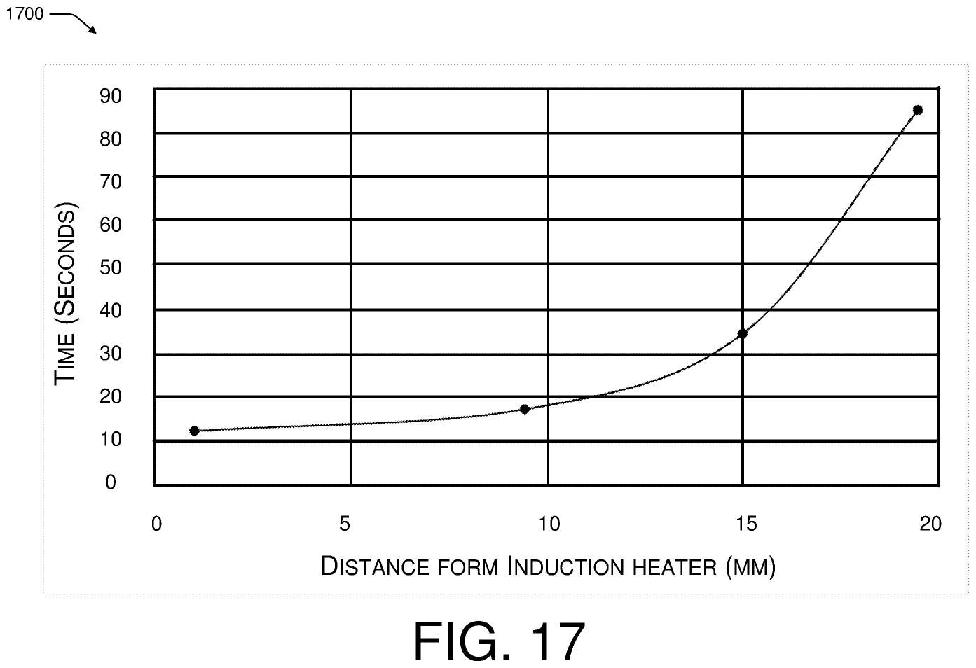

FIG. 17 illustrates a curve 1700 of the time to heat the regulating element to 100° C. versus the distance between the regulating element and the actuating component. The regulating element will melt even if the implantable valve is placed away (as far as 20 millimeters) from the actuating component, with a longer heating time.

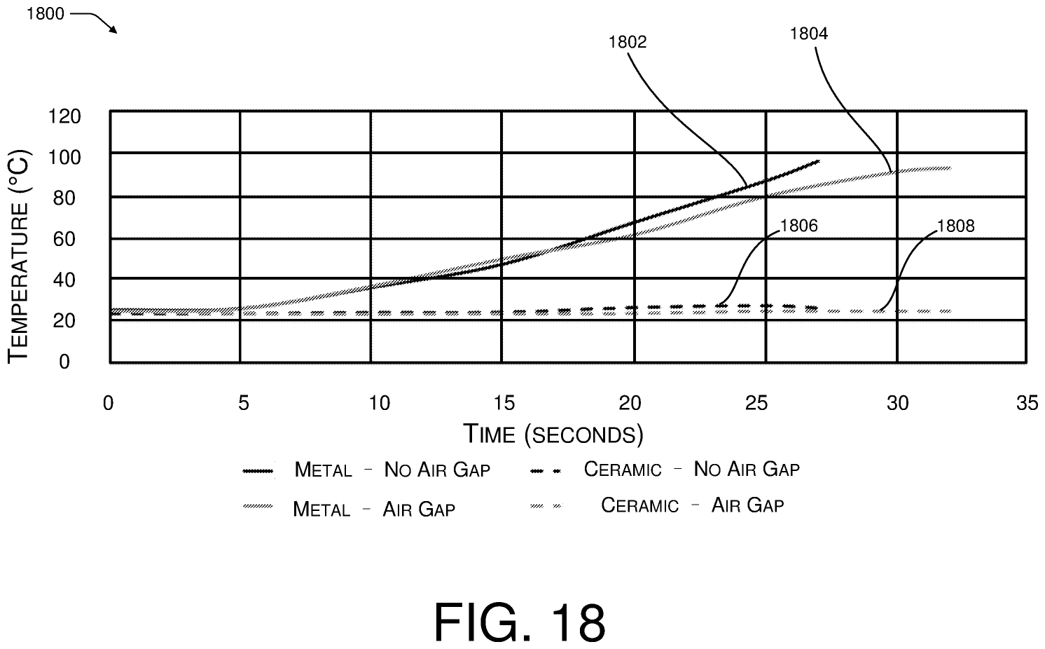

FIG. 18 illustrates a diagram 1800 showing curves of temperatures versus time. As shown in FIG. 18, curve 1802 shows the temperature of the valve interior versus time, where the heating element is made of metal and there is no air gap between the housing and the heating element. Curve 1804 shows the temperature of the valve interior versus time, where the heating element is made of metal and there is an air gap between the housing and the heating element. Curve 1806 shows the temperature of the valve exterior versus time, where the hosing is made of ceramic and there is no air gap between the hosing and the heating element. Curve 1808 shows the temperature of the valve exterior versus time, where the hosing is made of ceramic and there is an air gap between the hosing and the heating element.

As shown in FIG. 18, the interior temperature of the valve goes up, and the temperature of the valve exterior almost maintains the same without a significant rise. Therefore, the valve has the feature of insulating heat and is safe to be implanted into the human body.

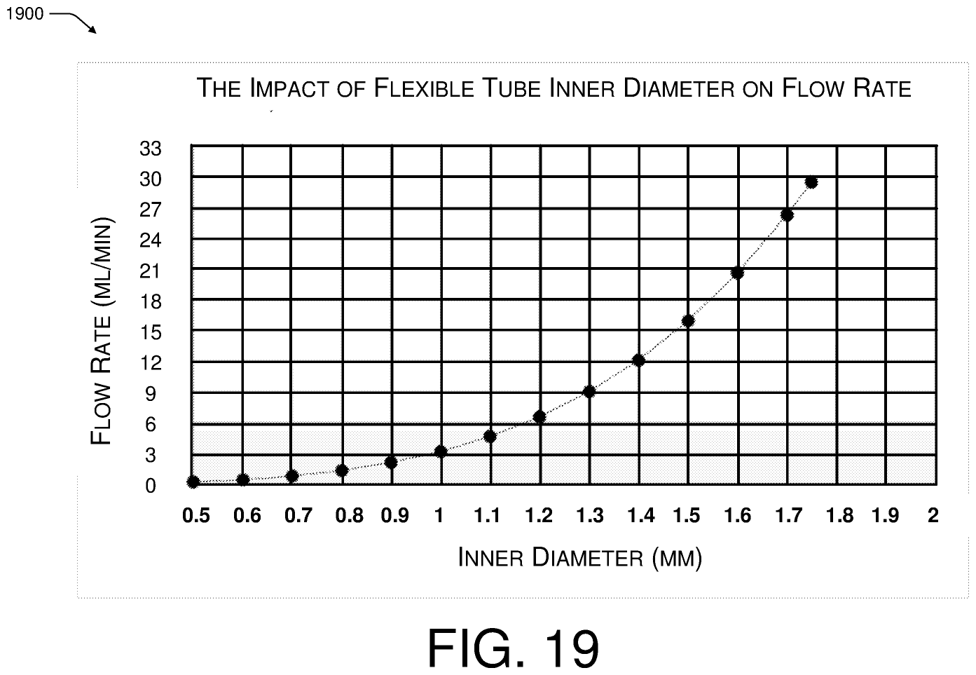

FIG. 19 illustrates the impact of the inner diameter of the tubing on the flow rate of the fluid flowing through the tubing. Referring to FIG. 19, curve 1900 shows a flow rate of the fluid flowing through the tubing versus the inner diameter of the tubing. As the inner diameter of the tubing increases, the flow rate of the fluid flowing through the tubing increases. The diameter of the flexible tube determines the valve's maximum output. The advantages of optimizing the flexible tube include, but are not limited to, reducing power consumption, achieving physiologic range, over-drainage protection, optimization of the valve's physical dimensions, and so on. Equation (1) can be used to design the tubing.

$$Δ p = \frac{8 μ LQ}{π {R}^{4}} \qquad ( 1 )$$

In Equation (1), Δp represents the pressure difference between the two ends of the valve; μ represents dynamic viscosity; L represents the length of the tubing; Q represents the volumetric flow rate of the fluid flowing through the tubing; and R represents the inner diameter of the tubing.

In implementations, the inner diameter of the tubing can be between 0.2 mm and 1.4 mm. The outer diameter of the tubing can be between 0.4 mm and 1.6 mm. The tubing can be made of flexible materials such as polyurethane, silicone, natural rubber, and so on. A stiff tubing requires a substantial amount of compression to close the valve, meaning that a higher temperature range is needed to close the valve.

### (XII) Methods and Operations of Controlling a System with Implantable Valve and External Actuating Component

Methods and operations described herein can be used to control a system including an implantable valve and an actuating component, such as an external actuating components as described above, to regulate biological fluid flow (e.g., CSF, or the like) or other fluid delivery into or out of the body of a patient. The implantable valve is suitable to be implanted into a patient's body. The actuating component, in embodiments, is suitable to be arranged outside the patient's body.

In some examples, methods, and operations described herein can be implemented to match the CSF outflow of a valve system to the CSF inflow (CSF production) associated with a patient. Activity-based changes in pressure such as a sneezing, coughing, and standing up will not impact the outflow rate of the valve. In some examples, methods, and operations described herein can be used to treat hydrocephalous patients, improving patients' quality of life in the short term by stopping over-drainage induced headaches and in the long term by reducing the shunt failure rate.

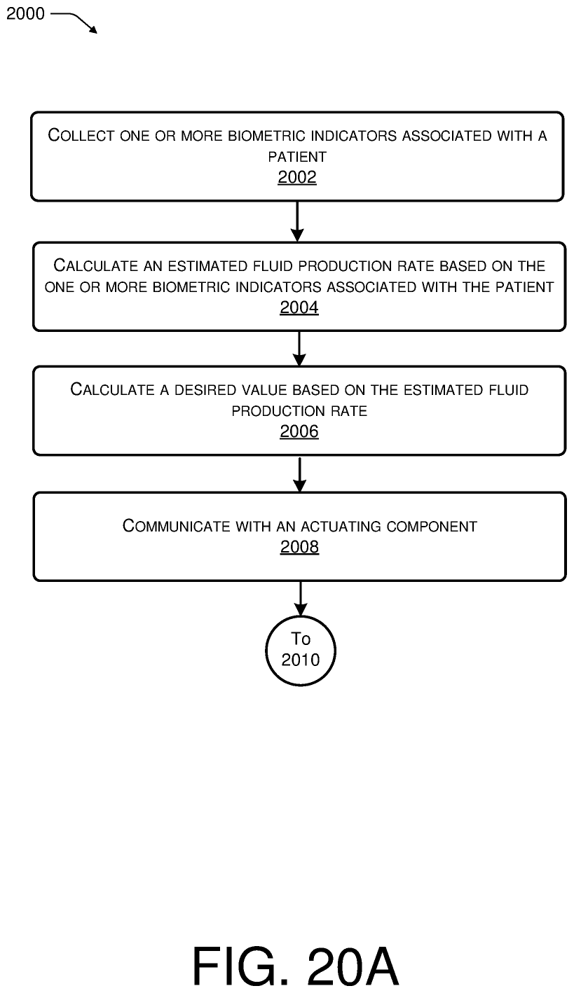

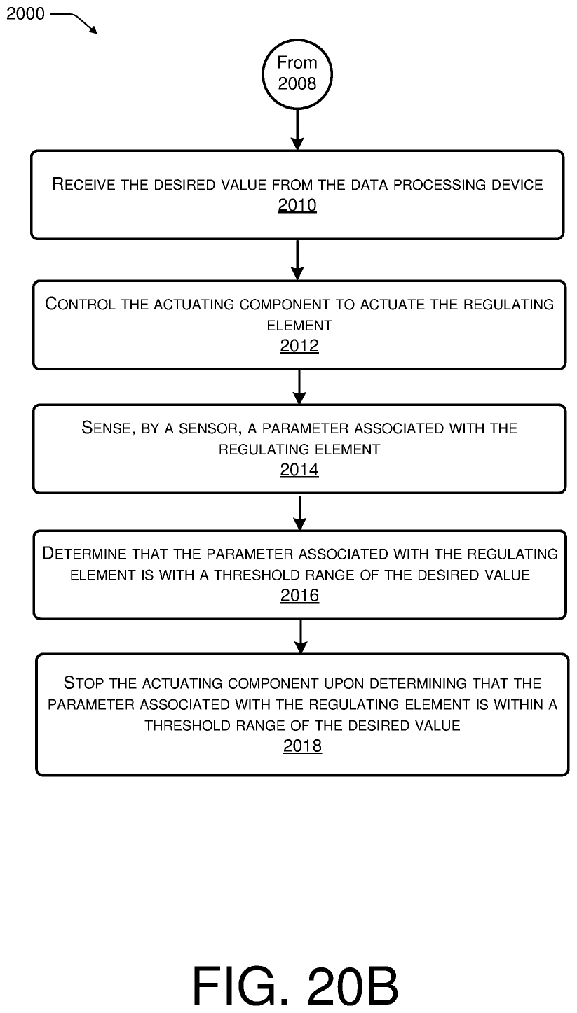

The example processes (e.g., in FIGS. 20A and 20B) are illustrated as logical flow graphs, each operation of which represents a sequence of operations that can be implemented in hardware, software, or a combination thereof. In the context of software, the operations represent computer-executable instructions stored on one or more computer-readable storage media that, when executed by one or more processors, configure a device to perform the recited operations. Generally, computer-executable instructions include routines, programs, objects, components, data structures, and the like that perform particular functions. The order in which the operations are described is not intended to be construed as a limitation, and any number of the described operations can be combined in any order and/or in parallel to implement the process. Further, any of the individual operations can be omitted, repeated, reordered, and/or combined with other operations.

FIG. 20A and FIG. 20B illustrate an example process 2000 for regulating biological fluid flow in accordance with implementations of this disclosure.

Referring to FIG. 20A, in 2002, operations include collecting, by a data processing device, one or more biometric indicators associated with a patient. In some examples, the one or more biometric indicators associated with the patient include a heart rate, a blood pressure, a posture indicator, an exercise indicator, and/or an endogenous brain pressure. The data processing device can correspond to the data processing device 1406 described with respect to FIG. 14.

At 2004, operations include calculating, by the data processing device, an estimated fluid production rate based on the one or more biometric indicators associated with the patient.

At 2006, operations include calculating, by the data processing device, a desired value based on the estimated fluid production rate. In some examples, the desired value includes a desired temperature.

At 2008, operations include communicating, by the data processing device, with an actuating component to actuate the regulating element of an implantable valve to regulate fluid flowing therethrough based on the desired value. The actuating component in some embodiments is suitable to be arranged outside the patient's body. Additionally, in some embodiments, the actuating component is suitable to be arranged inside the patient's body. The actuating component can correspond to the actuating component 1404 described with respect to FIG. 14. The implantable valve can correspond to the implantable valve 1300 described with respect to FIG. 13A and FIG. 13B, or the like. In some examples, the fluid flowing through the implantable valve includes biological fluids in organs or tissues, such as cerebrospinal fluid (CSF) in brain tissue, blood in the circulatory system, and so on. The implantable valve includes a housing, a tubing, a regulating element, an actuator, a sensor, a control component, and a power unit.

Referring to FIG. 20B, at 2010, operations include receiving, by a control component of the actuating component, the desired value from the data processing device.

At 2012, operations include controlling, by the control component of the actuating component, an electromagnetic field generator (e.g., a coil, a magnetic solenoid, or the like) of the actuating component to generate an electromagnetic field to actuate the regulating element of the implantable valve. In some examples, the regulating element includes wax and at least one additive (e.g., graphite, ferromagnetic material, and so on).

At 2014, operations include sensing, by a sensor in the implantable valve, a parameter associated with the regulating element. In some examples, the parameter associated with the regulating element is temperature associated with the regulating element.

At 2016, operations include determining, by the control component of the actuating component, that the parameter associated with the regulating element is within a threshold range of the desired value.

At 2018, operations include stopping, by the control component of the actuating component, the actuating component to strop generating the electromagnetic field upon determining that the parameter associated with the regulating element is within a threshold range of the desired value.

### (XIII) Example 1

Shunt valves and systems including the same were developed to regulate biological fluid flow such as CSF flow in a patient's brain. Such a system offered protection against activity-based changes in pressure, and was specifically engineered to improve patients' quality of life in the short term by stopping over-drainage induced headaches and in the long term by reducing the shunt failure rate. To validate whether the techniques described herein could work as expected, an in vitro system was established, and data were collected.

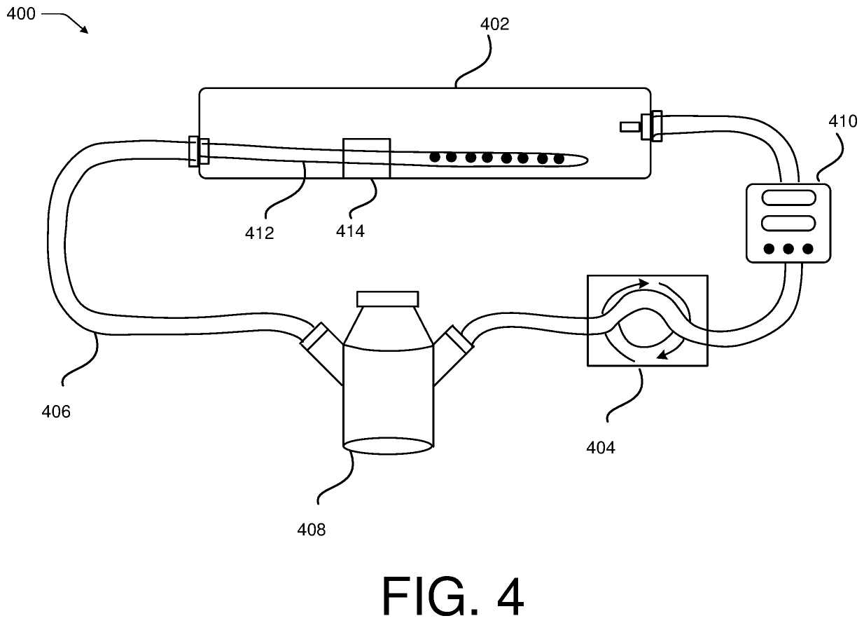

FIG. 4 illustrates an in vitro system 400 for testing and validating the valve and/or the system in accordance with implementations of this disclosure.

The in vitro system 400 was configured to include a chamber 402, a pump 404, a tube 406, a reservoir 408, and a meter 410. The pump 404 was configured to move the fluid through the tube 406 to the chamber 402. In some examples, the pump 404 was a peristatic pump. Note that other types of pumps can be used to push the fluid circulating in the vitro system 400, and this disclosure is not limited thereto. The reservoir 408 was configured to store the fluid. The tube 406 was connected between the chamber 402, the pump 404, the reservoir 408, and the meter 410. The meter 410 was configured to measure characteristics associated with the fluid such as the flowrate associated with the fluid, the frequency associated with the fluid, or the like. Note that other parameters/characteristics associated with the fluid can be measured by the meter 410.

In implementations, a catheter 412 to be tested and a valve 414 to be tested were placed in the chamber 402. The valve 414 was arranged around the catheter 412. The valve 414 was configured to regulate the fluid flowing through the catheter 412. In some examples, the valve 414 can correspond to the valve 100, the valve 100′, and/or the valve 100″ as described with respect to FIG. 1A, FIG. 1B, and FIG. 1C, the valve 202 described with respect to FIG. 2, or the like. In some examples, the in vitro system 400 can also be used to test conventional valves and/or catheters.

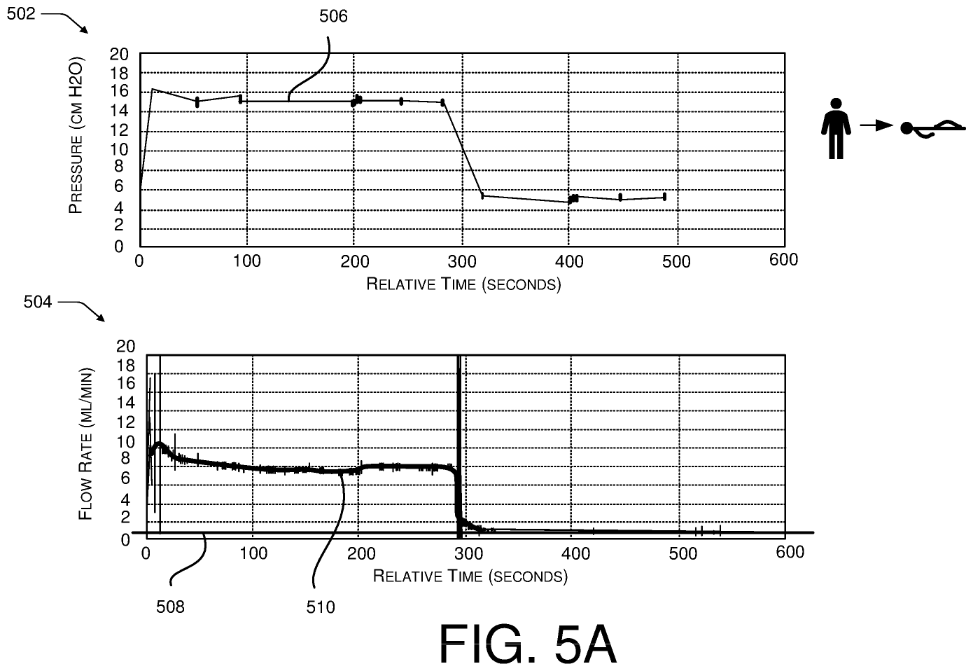

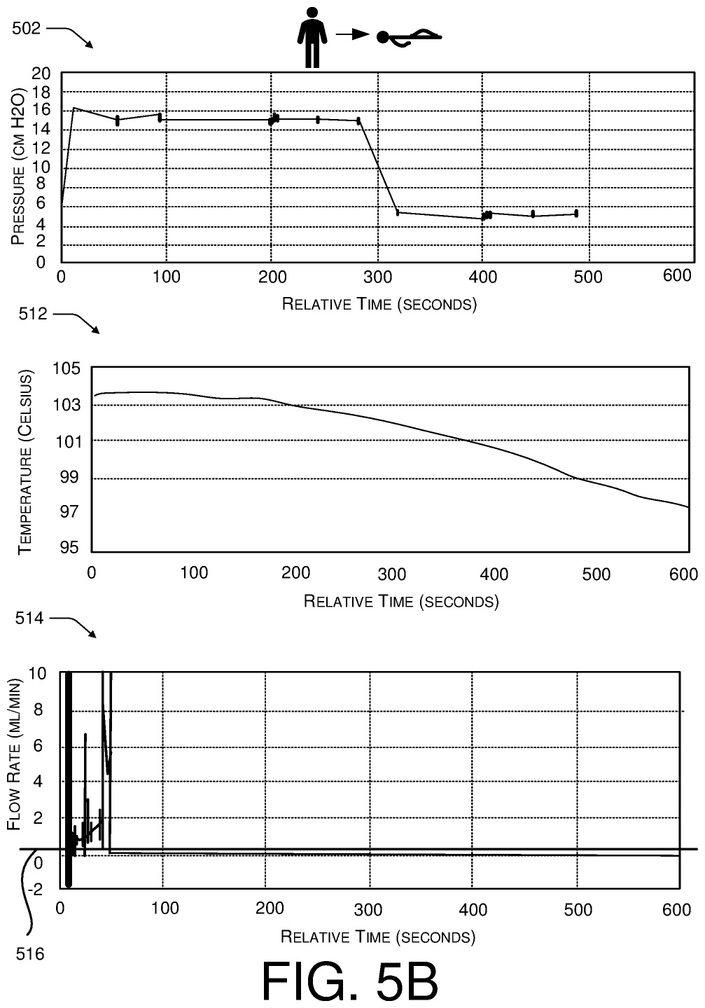

FIG. 5A and FIG. 5B illustrate graphs showing data when a patient changed position from standing up to laying down.

As described above, conventional shunt valve controlled CSF outflow by total pressure within the ventricles. The conventional shunt's one-way pressure valve was set to relieve pressure from the ventricles based off of total pressure, where total pressure=hydrostatic pressure+ (intracranial pressure (ICP)-intraabdominal pressure (IAP)). If the total pressure within the ventricles exceeds that of the conventional valve, the conventional valve will open. It will remain open unless the pressure drops below the set valve pressure. However, many events such as sneezing, coughing, straining from constipation, exercising, and standing up of the patient can cause changes in pressure, without increasing the production of the CSF. As a result, the conventional shunt valve can over-drain the CSF because it was pressure-based rather than matching the CSF production.

Referring to FIG. 5A, graph 502 shows the pressure change as a patient changed position from standing up to laying down. The horizontal axis in graph 502 represents relative time in seconds. The vertical axis in graph 502 represents the pressure in cm H₂O. Plot 506 represents the pressure along time. As shown in graph 502, the pressure changed at the time of 300 seconds when a patient changed position from standing up to laying down.

Graph 504 shows the flow rate change of a conventional shunt valve. The horizontal axis in graph 504 represents relative time in seconds. The vertical axis in graph 504 represents the flow rate in mL/min. In graph 504, line 508 represents the CSF production which was 0.3 mL/min in this example. Plot 510 represents the flow rate of the conventional shunt valve along time. As shown in graph 504, though the patient changed position from standing up to laying down at the time of 300 seconds, the CSF production (see 506) did not change. However, at the time of 300 seconds the flow rate (see 510) of the conventional shunt valve changed suddenly to match the pressure change (see 506). Therefore, over-drainage occurs during the period 0-300 seconds.

Referring to FIG. 5B, graph 512 shows the temperature of the regulating element. The horizontal axis in graph 512 represents relative time in seconds. The vertical axis in graph 512 represents the temperature in degree Celsius.

Graph 514 shows the flow rate change of a valve according to this disclosure. The horizontal axis in graph 514 represents relative time in seconds. The vertical axis in graph 514 represents the flow rate in mL/min. It can be seen that the flow rate of the valve according to this disclosure did not change because of the pressure change at the time of 300 seconds. Line 516 shows that CSF production which was 0.3 mL/min in this example. Though the patient changed her position from standing up to laying down at the time of 300 seconds, the CSF production (see 516) did not change. It can be seen that the flow rate of the valve according to this disclosure matched the CSF production (see 516) despite the pressure change at the time of 300 seconds. As pressure changed, the flow rate of the valve according to this disclosure did not change, because it was dependent on temperature of the regulating element rather than the pressure. Therefore, there was no over-drainage due to the position change of the patient.

The valve according to this disclosure was capable of controlling the CSF outflow by regulating the resistance of the tubing which was independent of the intracranial pressure. Tissue contact could be attenuated with the valve that minimizes over-drainage by matching the CSF outflow to the physiologic CSF production. The valve offered protection against activity-based changes in pressure, and thus could improve patients' quality of life in the short term by preventing over-drainage induced headache and in the long term by reducing the shunt failure rate.

Note that the position change of the patient discussed here was an example, and how the valve performs in other situations such as exercising (such as walking, running, lifting, or the like), sneezing, coughing, straining from constipation can be tested and the results incorporated into embodiments of the system.

### (XIV) References

Gluski et al., Fluids Barriers CNS 17 (1): 46, 2020 (doi: 10.1186/s12987-020-00211-6)
Horbatiuk et al., RSC Adv. 10 (52): 31056-30164, 2020 (doi: 10.1039/d0ra05128d)
Khodadadei et al., Commun. Biol. 4 (1): 387, 2021 (doi: 10.1038/s42003-021-01888-7)
Harris et al., Fluids Barriers CNS 18 (1): 4, 2021 (doi: 10.1186/s12987-021-00237-4)

### (XV) Example Clauses

CC: A device for regulating fluid flow, including: a tubing, configured to allow fluid flow therethrough; an implantable valve including a regulating element, arranged proximate to the tubing, the regulating element including ferromagnetic material; and an external actuating component, arranged proximate to the regulating element, the external actuating component being configured to manipulate a parameter associated with the regulating element, wherein the actuating component is configured to be arranged external to a subject's body or configured to be arranged inside the subject's body, wherein the external actuating component includes an electromagnetic field generator.

CD: The device of paragraph CC, configured to regulate fluid flowing from inside the subject's body to outside the subject's body.

CE: The device of paragraph CC, configured to regulate fluid flowing from outside the subject's body to inside the subject's body.

CF: A device for regulating fluid flow, including: a tubing, configured to allow fluid flow therethrough; an implantable valve including a regulating element, arranged proximate to the tubing; an actuating component, arranged proximate to the regulating element, the actuating component being configured to manipulate a parameter associated with the regulating element; and a control component, configured to communicate with other devices and to control the actuating component.

CG: The device of paragraph CF, wherein the actuating component is configured to be arranged external to the subject's body.

CH: The device of paragraph CF, wherein the actuating component is configured to be arranged inside to the subject's body.

CI: The device of paragraph CF, configured to regulate fluid flowing from inside the subject's body to outside the subject's body.

CJ: The device of paragraph CF, configured to regulate fluid flowing from outside the subject's body to inside the subject's body.

CK: The device of any one of clauses CC-CJ, further including a housing, configured to accommodate at least a part of the tubing and the implantable valve.

CL: The device of paragraph CK, wherein the housing further includes an insulating material, configured to reduce heat conduction between the housing and an environment.

CM: The device of any one of clauses CC-CJ, further including a sensor configured to sense the parameter associated with the regulating element.

CN: The device of CM, wherein the sensor includes at least one of a temperature sensor, a flowrate sensor, or a pressure sensor.

CO: The device of any one of clauses CF-CJ, wherein the control component is further configured to communicate with a data processing device, the data processing device being configured to collect one or more biometric indicators associated with a patient; and calculate an estimated fluid production rate based on the one or more biometric indicators associated with the patient; calculate a desired value based on the estimated fluid production rate; and send the desired value to the control component.

CP: The device of paragraph CO, wherein the one or more biometric indicators associated with the patient include a heart rate, a blood pressure, a posture indicator, an exercise indicator, and/or an endogenous brain pressure.

CQ: The device of paragraph CO, wherein the control component is further configured to receive the desired value from the data processing device.

CR: The device of paragraph CO, wherein the desired value includes a desired temperature.

CS: The device of any one of clauses CF-CJ, wherein the control component is further configured to communicate with the other devices via Bluetooth or other wireless manners.

CT: The device of any one of clauses CC-CJ, wherein the regulating element includes wax and at least one additive.

CU: The device of any one of clauses CT, wherein the additive includes ferromagnetic material.

CV: The device of any one of clauses CC-CJ, wherein the regulating element is configured to regulate resistance to the fluid flowing through the tubing, wherein the resistance is independent of intracranial pressure associated with a patient.

CW: The device of any one of clauses CC-CJ, wherein the parameter associated with the regulating element is temperature associated with the regulating element.

CX: The device of any one of clauses CC-CJ, wherein the external actuating component includes an electromagnetic field generator.

CY: The device of any one of clauses CX, further including a power unit configured to provide power for the electromagnetic actuating component.

CZ: The device of paragraph CY, wherein the power unit includes a Lithium battery.

DA: The device of any one of clauses CC-CZ, wherein the fluid includes cerebrospinal fluid (CSF).

DB: A system for regulating fluid flow, including: a data processing device, configured to collect one or more biometric indicators associated with a patient; and a tubing, configured to allow fluid flow therethrough; an implantable valve including a regulating element, arranged proximate to the tubing; an external actuating component, arranged proximate to the regulating element, the actuating component being configured to manipulate a parameter associated with the regulating element, wherein the actuating component is configured to be arranged external to a subject's body; and a control component, configured to communicate with other devices and to control the external actuating component.

DC: The system of paragraph DB, configured to regulate fluid flowing from inside the subject's body to outside the subject's body.

DD: The system of paragraph DB, configured to regulate fluid flowing from outside the subject's body to inside the subject's body.

DE: The system of paragraph DB, wherein the data processing device includes a wearable device.

DF: The system of paragraph DB, wherein the data processing device is further configured to calculate an estimated fluid production rate based on the one or more biometric indicators associated with the patient; calculate a desired value based on the estimated fluid production rate; and send the desired value to the control component.

DG: The system of paragraph DB, wherein the desired value includes a desired temperature.

DH: The system of paragraph DB, wherein the one or more biometric indicators associated with the patient include a heart rate, a blood pressure, a posture indicator, an exercise indicator, and/or an endogenous brain pressure.

DI: The system of paragraph DB, wherein the implantable valve further includes a housing, configured to accommodate the tubing and the regulating element.

DJ: The system of paragraph DI, wherein the housing further includes an insulating material, configured to reduce heat conduction between the housing and an environment.

DK: The system of paragraph DB, wherein the implantable valve further includes a sensor configured to sense the parameter associated with the regulating element.

DL: The system of paragraph DK, wherein the sensor includes at least one of a temperature sensor, a flowrate sensor, or a pressure sensor.

DM: The system of paragraph DB, wherein the regulating element includes wax and at least one additive.

DN: The system of paragraph DM, wherein the additive includes wherein the additive includes ferromagnetic material.

DO: The system of paragraph DB, wherein the regulating element is configured to regulate resistance to the fluid flowing through the tubing, wherein the resistance is independent of intracranial pressure associated with a patient.

DP: The system of paragraph DB, wherein the parameter associated with the regulating element is temperature associated with the regulating element.

DQ: The system of paragraph DB, wherein the external actuating component includes an electromagnetic field generator.

DR: The system of paragraph DB, further including a power unit configured to provide power for the external actuating component.

DS: The system of paragraph DR, wherein the power unit includes a Lithium battery.

DT: The system of paragraph DB, wherein the implantable valve further includes a heating element configured to heat the regulating element.

DU: The system of any one of clauses DB-DT, wherein the fluid includes cerebrospinal fluid (CSF).

DV: A method for regulating fluid flow, including: collecting one or more biometric indicators associated with a patient; calculating an estimated fluid production rate based on the one or more biometric indicators associated with the patient; calculating a desired value based on the estimated fluid production rate; and sending the desired value to a control component of an external actuating component for actuating an implantable valve of a device for regulating fluid flow, wherein the external actuating component is configured to be arranged external to the patient's body, the device for regulating fluid flow including a tubing, configured to allow fluid flow therethrough; and the implantable valve including a regulating element, arranged proximate to the tubing.

DW: The method of paragraph DV, wherein the device is configured to regulate fluid flowing from inside the subject's body to outside the subject's body.

DX: The method of paragraph DV, wherein device is configured to regulate fluid flowing from outside the subject's body to inside the subject's body.

DY: The method of paragraph DV, wherein the one or more biometric indicators associated with the patient include a heart rate, a blood pressure, a posture indicator, an exercise indicator, and/or an endogenous brain pressure.

EJ: The method of paragraph DV, wherein the desired value includes a desired temperature.

EK: The method of paragraph DV, further including: receiving, by the control component of the external actuating component, the desired value from the data processing device; controlling, by the control component of the external actuating component, the external actuating component to actuate the regulating element; sensing, by a sensor associated with the implantable valve, a parameter associated with the regulating element; determining, by the control component, that the parameter associated with the regulating element is within a threshold range of the desired value; and stopping, by the control component, the external actuating component upon determining that the parameter associated with the regulating element is within a threshold range of the desired value.

EL: The method of any one of clauses DV-EK, wherein the fluid includes cerebrospinal fluid (CSF).

EM: A computer-readable storage medium storing computer-readable instructions executable by one or more processors, that when executed by the one or more processors, causes the one or more processors to perform acts including: collecting one or more biometric indicators associated with a patient; calculating an estimated fluid production rate based on the one or more biometric indicators associated with the patient; calculating a desired value based on the estimated fluid production rate; and sending the desired value to a control component controlling an external actuating component for actuating an implantable valve of a device, wherein the external actuating component is configured to be arranged external to the patient's body, the device for regulating fluid flow including a tubing, configured to allow fluid flow therethrough; and the implantable valve including a regulating element, arranged proximate to the tubing.

EN: The computer-readable storage medium of paragraph EM, wherein the device is configured to regulate fluid flowing from inside the subject's body to outside the subject's body.

EO: The computer-readable storage medium of paragraph EM, wherein device is configured to regulate fluid flowing from outside the subject's body to inside the subject's body.

EP: The computer-readable storage medium of paragraph EM, wherein the one or more biometric indicators associated with the patient include a heart rate, a blood pressure, a posture indicator, an exercise indicator, and/or an endogenous brain pressure.

EQ: The computer-readable storage medium of paragraph EM, wherein the desired value includes a desired temperature.

ER: The computer-readable storage medium of paragraph EM, wherein the parameter associated with the regulating element is temperature of the regulating element.

ES: The computer-readable storage medium of paragraph EM, the operations further including: receiving, by the control component controlling the external actuating component, the desired value from the data processing device; controlling, by the control component, the external actuating component to actuate the regulating element; sensing, by a sensor associated with the implantable valve, a parameter associated with the regulating element; determining, by the control component, that the parameter associated with the regulating element is within a threshold range of the desired value; and stopping, by the control component, the external actuating component upon determining that the parameter associated with the regulating element is within a threshold range of the desired value.

ET: The computer-readable storage medium any one of clauses EM-ES, wherein the fluid includes cerebrospinal fluid (CSF).

### (XVI) Closing Paragraphs

Specific descriptions provided herein and in the herewith filed documents are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references have been made to patents, printed publications, journal articles and other written text throughout this specification (referenced materials herein). Each of the referenced materials is individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A device for regulating fluid flow, comprising:
a tubing, configured to allow fluid flow therethrough;
an implantable valve comprising a regulating element, arranged proximate to the tubing, the regulating element comprising ferromagnetic material; and
an external actuating component, arranged proximate to the regulating element, the external actuating component being configured to manipulate a parameter associated with the regulating element, wherein the actuating component is configured to be arranged external to a subject's body or configured to be arranged inside the subject's body, wherein the external actuating component comprises an electromagnetic field generator.

2. The device of claim 1, configured to regulate fluid flowing from inside the subject's body to outside the subject's body.

3. The device of claim 1, configured to regulate fluid flowing from outside the subject's body to inside the subject's body.

4. A device for regulating fluid flow, comprising:
a tubing, configured to allow fluid flow therethrough;
an implantable valve comprising a regulating element, arranged proximate to the tubing;
an actuating component, arranged proximate to the regulating element, the actuating component being configured to manipulate a parameter associated with the regulating element; and
a control component, configured to communicate with other devices and to control the actuating component.

5. The device of claim 4, wherein the actuating component is configured to be arranged external to the subject's body.

6. The device of claim 4, wherein the actuating component is configured to be arranged inside to the subject's body.

7. The device of claim 4, configured to regulate fluid flowing from inside the subject's body to outside the subject's body.

8. The device of claim 4, configured to regulate fluid flowing from outside the subject's body to inside the subject's body.

9. The device of any one of claims 1-8, further comprising a housing, configured to accommodate at least a part of the tubing and the implantable valve.

10. The device of claim 9, wherein the housing further comprises an insulating material, configured to reduce heat conduction between the housing and an environment.

11. The device of any one of claims 1-8, further comprising a sensor configured to sense the parameter associated with the regulating element.

12. The device of 11, wherein the sensor comprises at least one of a temperature sensor, a flowrate sensor, or a pressure sensor.

13. The device of any one of claims 4-8, wherein the control component is further configured to communicate with a data processing device, the data processing device being configured to
collect one or more biometric indicators associated with a patient; and
calculate an estimated fluid production rate based on the one or more biometric indicators associated with the patient;
calculate a desired value based on the estimated fluid production rate; and
send the desired value to the control component.

14. The device of claim 13, wherein the one or more biometric indicators associated with the patient include a heart rate, a blood pressure, a posture indicator, an exercise indicator, and/or an endogenous brain pressure.

15. The device of claim 13, wherein the control component is further configured to receive the desired value from the data processing device.

16. The device of claim 13, wherein the desired value comprises a desired temperature.

17. The device of any one of claims 4-8, wherein the control component is further configured to communicate with the other devices via Bluetooth or other wireless manners.

18. The device of any one of claims 1-8, wherein the regulating element comprises wax and at least one additive.

19. The device of any one of claim 18, wherein the additive comprises ferromagnetic material.

20. The device of any one of claims 1-8, wherein the regulating element is configured to regulate resistance to the fluid flowing through the tubing, wherein the resistance is independent of intracranial pressure associated with a patient.

21. The device of any one of claims 1-8, wherein the parameter associated with the regulating element is temperature associated with the regulating element.

22. The device of any one of claims 1-8, wherein the external actuating component comprises an electromagnetic field generator.

23. The device of any one of claim 22, further comprising a power unit configured to provide power for the electromagnetic actuating component.

24. The device of claim 23, wherein the power unit comprises a Lithium battery.

25. The device of any one of claims 1-24, wherein the fluid comprises cerebrospinal fluid (CSF).

26. A system for regulating fluid flow, comprising:
a data processing device, configured to collect one or more biometric indicators associated with a patient; and
a tubing, configured to allow fluid flow therethrough;
an implantable valve comprising a regulating element, arranged proximate to the tubing;
an external actuating component, arranged proximate to the regulating element, the actuating component being configured to manipulate a parameter associated with the regulating element, wherein the actuating component is configured to be arranged external to a subject's body; and
a control component, configured to communicate with other devices and to control the external actuating component.

27. The system of claim 26, configured to regulate fluid flowing from inside the subject's body to outside the subject's body.

28. The system of claim 26, configured to regulate fluid flowing from outside the subject's body to inside the subject's body.

29. The system of claim 26, wherein the data processing device comprises a wearable device.

30. The system of claim 26, wherein the data processing device is further configured to
calculate an estimated fluid production rate based on the one or more biometric indicators associated with the patient;
calculate a desired value based on the estimated fluid production rate; and
send the desired value to the control component.

31. The system of claim 26, wherein the desired value comprises a desired temperature.

32. The system of claim 26, wherein the one or more biometric indicators associated with the patient include a heart rate, a blood pressure, a posture indicator, an exercise indicator, and/or an endogenous brain pressure.

33. The system of claim 26, wherein the implantable valve further comprises a housing, configured to accommodate the tubing and the regulating element.

34. The system of claim 33, wherein the housing further comprises an insulating material, configured to reduce heat conduction between the housing and an environment.

35. The system of claim 26, wherein the implantable valve further comprises a sensor configured to sense the parameter associated with the regulating element.

36. The system of claim 35, wherein the sensor comprises at least one of a temperature sensor, a flowrate sensor, or a pressure sensor.

37. The system of claim 26, wherein the regulating element comprises wax and at least one additive.

38. The system of claim 37, wherein the additive includes wherein the additive comprises ferromagnetic material.

39. The system of claim 26, wherein the regulating element is configured to regulate resistance to the fluid flowing through the tubing, wherein the resistance is independent of intracranial pressure associated with a patient.

40. The system of claim 26, wherein the parameter associated with the regulating element is temperature associated with the regulating element.

41. The system of claim 26, wherein the external actuating component comprises an electromagnetic field generator.

42. The system of claim 26, further comprising a power unit configured to provide power for the external actuating component.

43. The system of claim 42, wherein the power unit comprises a Lithium battery.

44. The system of claim 26, wherein the implantable valve further comprises a heating element configured to heat the regulating element.

45. The system of any one of claims 26-44, wherein the fluid comprises cerebrospinal fluid (CSF).

46. A method for regulating fluid flow, comprising:
collecting one or more biometric indicators associated with a patient;
calculating an estimated fluid production rate based on the one or more biometric indicators associated with the patient;
calculating a desired value based on the estimated fluid production rate; and
sending the desired value to a control component of an external actuating component for actuating an implantable valve of a device for regulating fluid flow, wherein the external actuating component is configured to be arranged external to the patient's body, the device for regulating fluid flow comprising
a tubing, configured to allow fluid flow therethrough; and
the implantable valve including a regulating element, arranged proximate to the tubing.

47. The method of claim 46, wherein the device is configured to regulate fluid flowing from inside the subject's body to outside the subject's body.

48. The method of claim 46, wherein device is configured to regulate fluid flowing from outside the subject's body to inside the subject's body.

49. The method of claim 46, wherein the one or more biometric indicators associated with the patient include a heart rate, a blood pressure, a posture indicator, an exercise indicator, and/or an endogenous brain pressure.

60. The method of claim 46, wherein the desired value comprises a desired temperature.

61. The method of claim 46, further comprising:
receiving, by the control component of the external actuating component, the desired value from the data processing device;
controlling, by the control component of the external actuating component, the external actuating component to actuate the regulating element;
sensing, by a sensor associated with the implantable valve, a parameter associated with the regulating element;
determining, by the control component, that the parameter associated with the regulating element is within a threshold range of the desired value; and
stopping, by the control component, the external actuating component upon determining that the parameter associated with the regulating element is within a threshold range of the desired value.

62. The method of any one of claims 46-61, wherein the fluid comprises cerebrospinal fluid (CSF).

63. A computer-readable storage medium storing computer-readable instructions executable by one or more processors, that when executed by the one or more processors, causes the one or more processors to perform acts comprising:
collecting one or more biometric indicators associated with a patient;
calculating an estimated fluid production rate based on the one or more biometric indicators associated with the patient;
calculating a desired value based on the estimated fluid production rate; and
sending the desired value to a control component controlling an external actuating component for actuating an implantable valve of a device, wherein the external actuating component is configured to be arranged external to the patient's body, the device for regulating fluid flow comprising
a tubing, configured to allow fluid flow therethrough; and
the implantable valve including a regulating element, arranged proximate to the tubing.

64. The computer-readable storage medium of claim 63, wherein the device is configured to regulate fluid flowing from inside the subject's body to outside the subject's body.

65. The computer-readable storage medium of claim 63, wherein device is configured to regulate fluid flowing from outside the subject's body to inside the subject's body.

66. The computer-readable storage medium of claim 63, wherein the one or more biometric indicators associated with the patient include a heart rate, a blood pressure, a posture indicator, an exercise indicator, and/or an endogenous brain pressure.

67. The computer-readable storage medium of claim 63, wherein the desired value comprises a desired temperature.

68. The computer-readable storage medium of claim 63, wherein the parameter associated with the regulating element is temperature of the regulating element.

69. The computer-readable storage medium of claim 63, the operations further comprising:
receiving, by the control component controlling the external actuating component, the desired value from the data processing device;
controlling, by the control component, the external actuating component to actuate the regulating element;
sensing, by a sensor associated with the implantable valve, a parameter associated with the regulating element;
determining, by the control component, that the parameter associated with the regulating element is within a threshold range of the desired value; and
stopping, by the control component, the external actuating component upon determining that the parameter associated with the regulating element is within a threshold range of the desired value.

70. The computer-readable storage medium any one of claims 63-69, wherein the fluid comprises cerebrospinal fluid (CSF).
